(19) United States
(12) Patent Application Publication
(10) Pub. No.: US 2006/0246133 A1
(43) Pub. Date: Nov. 2, 2006

(54) Controlled release pharmaceutical compositions of liothyronine and methods of making and using the same

(76) Inventors: Martin Wade Beasley, Cary, NC (US); David Paul Hause, Carrboro, NC (US); Irwin Klein, Manhasset, NY (US); Charles L. Pamplin, Chapel Hill, NC (US); David John Reynolds, Durham, NC (US); Kevin Hugh Sills, Apex, NC (US)

Correspondence Address: JONES DAY, 222 EAST 41ST ST, NEW YORK, NY, 10017, US

(21) Appl. No.: 11/396,420

(22) Filed: Mar. 31, 2006

Related U.S. Application Data
(60) Provisional application No. 60/666,621, filed on Mar. 31, 2005.

(51) Int. Cl. A61K 31/198; A61K 9/22

(52) U.S. Cl. 424/468; 514/567

(57) ABSTRACT

This invention relates to sustained release pharmaceutical compositions comprising liothyronine, or a salt or derivative thereof. Additionally, the present invention is directed to methods of manufacture and methods of using the pharmaceutical compositions of the present invention.

This application claims the benefit of priority of U.S. Provisional Application Ser. No. 60/666,621, filed Mar. 31, 2005; the disclosure of which is hereby incorporated by reference in its entirety.

### 1. FIELD OF INVENTION

The present invention relates generally to controlled release pharmaceutical compositions. Specifically, the present invention relates to controlled release pharmaceutical compositions comprising liothyronine, or a salt or derivative thereof. Additionally, the present invention is directed to methods of manufacture and methods of using the pharmaceutical compositions of the present invention.

### 2. BACKGROUND

More than eight million Americans suffer from hypothyroidism. Hypothyroidism occurs when the thyroid gland produces insufficient amounts of thyroid hormone. Low levels of thyroid hormone can result in a slower metabolism rate, causing an individual to feel cold, run down, sluggish, and tired. Low levels of thyroid hormone can also cause hair to become brittle and skin to become dry and itchy.

It is estimated that 17% of women and 8% of men, who are 60 years of age or older, suffer from hypothyroidism. The most common cause of low thyroid production is an autoimmune disease called Hashimoto's Thyroiditis which occurs when lymphocytes make antibodies which slowly and gradually disable the hormone-producing cells in the thyroid gland. Hypothyroidism can also be caused by deficient levels of iodine in the body. For example, diets low in iodine can contribute to the development of hypothyroidism and the many serious physical and mental problems associated with it.

Unfortunately, hypothyroidism is frequently under diagnosed even though there is a simple blood test that measures the amount of thyroid stimulating hormone (TSH) in the body. A high TSH indicates that the thyroid gland is not producing sufficient amounts of thyroid hormone.

Once properly diagnosed, however, treatment is straightforward. The missing thyroid hormone is replaced with thyroxine (T4), currently available in tablet dosage form. For many patients, however, administration of thyroxine alone is insufficient, due to the body's limited capacity to convert thyroxine to liothyronine (T3), which is biologically more active than thyroxine. Research suggests that for such individuals a mixture of thyroid hormones thyroxine and liothyronine maybe a more effective form of treatment than thyroxine alone.

Currently, liothyronine is available in an immediate release form under the name Cytomel® (King Pharmaceuticals, Inc. Bristol, Tenn.). The use of Cytomel®, however, is not without its drawbacks. For example, administration of Cytomel® results in an undesired, initial, acute plasma level peak of liothyronine. Such an abrupt change in the plasma level of liothyronine can cause adverse, short-term side effects such as increased heart rate, nervousness, anxiousness and irritability and long-term side effects such as a decrease in bone density. Also, when administered in an immediate release form liothyronine has a half-life of about 10 hours and, therefore, must be administered twice daily. The twice daily administration places an added burden on patients and exposes the patient to two undesired initial, acute plasma level peaks of liothyronine.

Therefore, a controlled release pharmaceutical composition provides many advantages over conventional immediate release pharmaceutical compositions. The advantages include less frequent dosing, increased patient compliance, a more sustained drug blood level response, therapeutic action with less ingested drug and fewer side effects. By providing a slow and steady release of the medicament over time by use of a controlled release composition, absorbed concentration peaks are mitigated or even eliminated by effecting smoother and more sustained blood level response.

Thus, it is desirable in the treatment of hypothyroidism, as well as other diseases, both therapeutically and prophylactically, to provide a biologically active material, preferably one suitable for the treatment of hypothyroidism, in a controlled release form which provides a controlled rate of release of a medicament over an extended period.

### 3. SUMMARY

3.1. Definitions

As used herein, and unless otherwise indicated, the phrase “baseline concentration” means the circulating endogenous concentration of liothyronine in a subject immediately prior to the administration of the sustained release pharmaceutical compositions of the present invention.

As used herein, and unless otherwise indicated, the terms “controlled release”, “sustained release” and “modified release” can be used interchangeably and are used to describe pharmaceutical compositions of the present invention wherein the release of the active pharmaceutical ingredient (API) is such that an immediate, acute plasma level peak is mitigated or eliminated as compared to immediate release pharmaceutical compositions of the same drug.

As used herein, and unless otherwise indicated, the terms “individual”, “subject” or “patient” can be used interchangeably and are not limited to an individual under the care of a physician.

As used herein, and unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

The term “pharmaceutically acceptable salts” refers to salts prepared from pharmaceutically acceptable non-toxic acids or bases including inorganic acids and bases and organic acids and bases. Suitable pharmaceutically acceptable base addition salts include, but are not limited to, metallic salts made from aluminum, calcium, lithium, magnesium, potassium, sodium and zinc and organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences (18th ed., Mack Publishing, Easton Pa.: 1990) and Remington: The Science and Practice of Pharmacy (19th ed., Mack Publishing, Easton Pa.: 1995).

As used herein, and unless otherwise specified, the phrase “optimal plasma level concentration”, means a plasma level concentration of liothyronine wherein the subject no longer suffers from hypothyroidism or the symptoms associated with hypothyroidism. The optimal plasma level concentration will vary by subject and will depend, in large part, on the age, height, weight, and sex of the subject. In general, however, when testing for or monitoring hypothyroidism a TSH range between 0.5 to 5.0 uIU/ml is likely to indicate optimal plasma level concentrations of liothyronine.

As used herein, and unless otherwise specified, a “prophylactically effective amount” or “therapeutically effective amount” can be used interchangeably and mean an amount of a compound sufficient to prevent a disease or condition, or one or more symptoms associated with the disease or condition, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

As used herein, unless otherwise indicated, the terms “prevent,” “preventing” and “prevention” contemplate an action that occurs before a patient begins to suffer from the specified disease or disorder, which inhibits or reduces the severity of the disease or disorder.

As used herein, and unless otherwise specified, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound means an amount of therapeutic agent, alone or in combination with other therapies, which provides a therapeutic benefit in the treatment or management of the disease or condition. The term “therapeutically effective amount” can encompass an amount that improves overall therapy, reduces or eliminates symptoms or causes of a disease or condition, or enhances the therapeutic efficacy of another therapeutic agent.

The terms “treat,” “treating” and “treatment,” as used herein, contemplate an action that occurs while a patient is suffering from the specified disease or disorder, which reduces the severity of the disease or disorder, or retards or slows the progression of the disease or disorder.

The present invention is directed to controlled release pharmaceutical compositions comprising liothyronine, or a pharmaceutically acceptable salt thereof that are capable of releasing liothyronine so as to eliminate, or at least mitigate, initial, acute liothyronine plasma concentration peaks. The present invention is also directed to controlled release pharmaceutical compositions comprising liothyronine, or a pharmaceutically acceptable salt thereof, that can release liothyronine so as to reduce or effectively eliminate undesirable liothyronine plasma level fluctuations. The present invention is further directed to controlled release pharmaceutical compositions comprising liothyronine, or a pharmaceutically acceptable salt thereof that can release liothyronine so as to maintain a steady state concentration of liothyronine.

Additionally, the present invention is directed to controlled release pharmaceutical compositions comprising liothyronine, or a pharmaceutically acceptable salt thereof that can release liothyronine so as to reduce the frequency or eliminate the occurrence of undesirable side effects, such as adverse cardiac effects.

The inventors have made the surprising discovery that by incorporating liothyronine, or a pharmaceutically acceptable salt thereof, into a rate-limiting matrix, the release of liothyronine can be controlled so as to eliminate, or at least mitigate, initial, acute plasma level peaks as well as, reduce the frequency or eliminate the occurrence of undesirable side effects associated with immediate release liothyronine formulations. Also by incorporating liothyronine, or a pharmaceutically acceptable salt thereof, into a rate-limiting matrix, the release of liothyronine can be controlled so as to reduce or eliminate plasma level fluctuations of liothyronine and maintain a steady-state concentration of liothyronine

In particular, the inventors have demonstrated that by incorporating liothyronine into a rate-limiting matrix the release of the liothyronine can be controlled so that initial, acute peak plasma levels of liothyronine are mitigated as compared to currently available immediate release compositions. Additionally, the inventors have discovered that by incorporating liothyronine, or a pharmaceutical salt thereof, in a rate-limiting matrix the maximum plasma concentration (“Cₘₐₓ”) is delayed as compared to currently available immediate release compositions.

As such, the pharmaceutical compositions contemplated by the present invention comprise liothyronine, or a pharmaceutical salt thereof, and at least one pharmaceutically acceptable excipient wherein the pharmaceutical composition is capable of mitigating or eliminating the initial, acute concentration peak of liothyronine characteristic of current immediate release liothyronine compositions as well as delay the occurrence of the maximum level of liothyronine concentration (“Cₘₐₓ”). As such, the contemplated compositions can also prolong the time taken to reach Cₘₐₓ (“Tₘₐₓ”) as compared to currently available immediate release compositions. Moreover, the pharmaceutical compositions of the present invention comprise liothyronine, or a pharmaceutical salt thereof, and at least one pharmaceutically acceptable excipient wherein the pharmaceutical composition mitigates or eliminates fluctuations in the plasma levels of thyroid hormone over time.

Specifically, the inventors have shown that the compositions of the present invention have improved bioavailability compared to Cytomel®. Additionally, the pharmaceutical compositions of the present invention allow liothyronine to maintain potency, assuring health care providers and patients that they are giving and receiving consistent and exact treatment.

The present invention also relates to methods of treating thyroid deficiency by administering a pharmaceutical composition of the present invention.

### 4. BRIEF DESCRIPTION

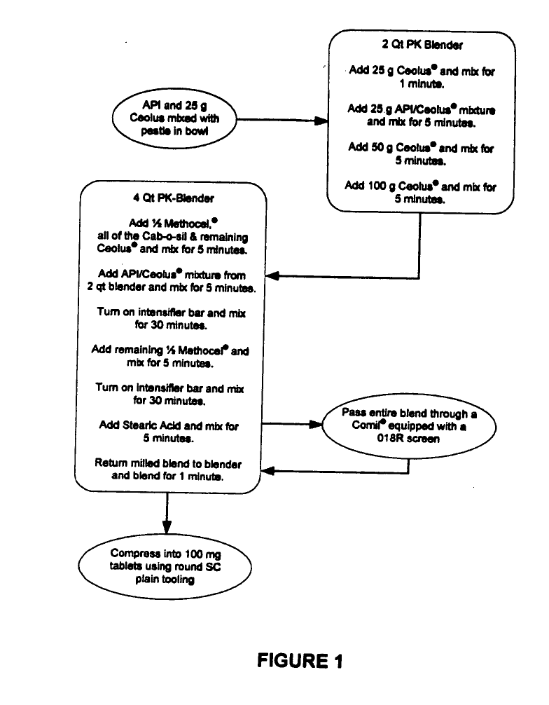

FIG. 1 shows a manufacturing flow chart.

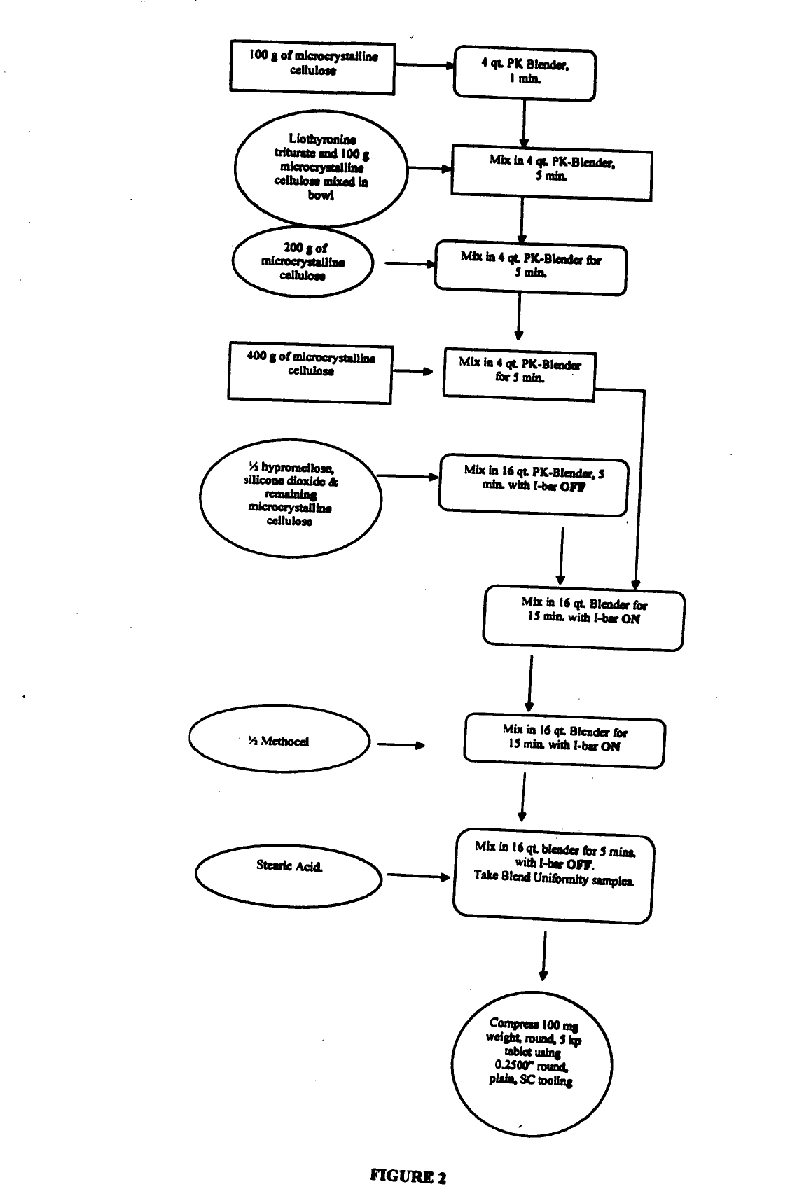

FIG. 2 shows a manufacturing flow chart.

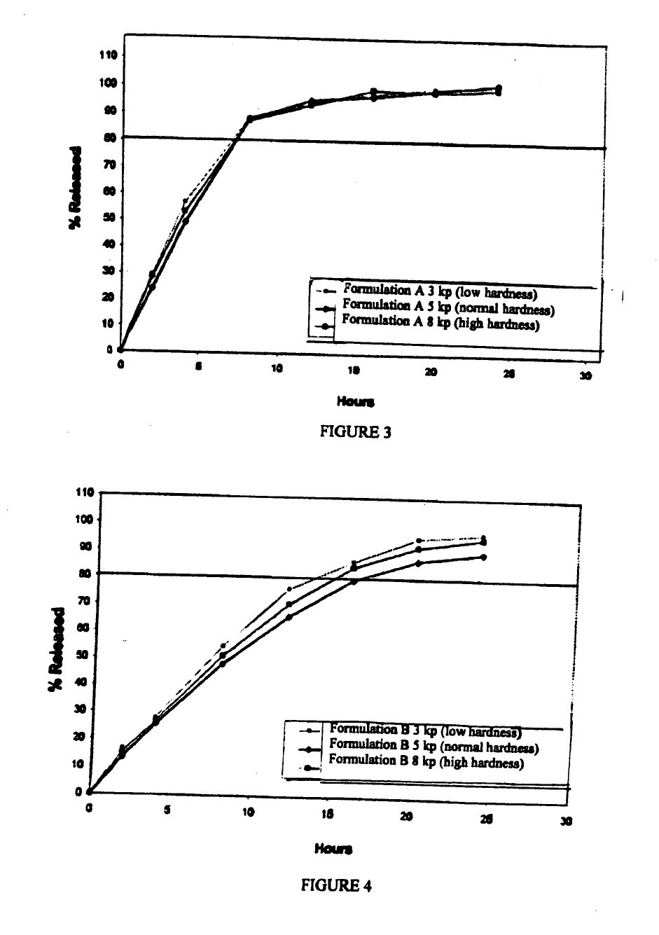

FIG. 3 shows dissolution profile information for tablets made from Formulation A.

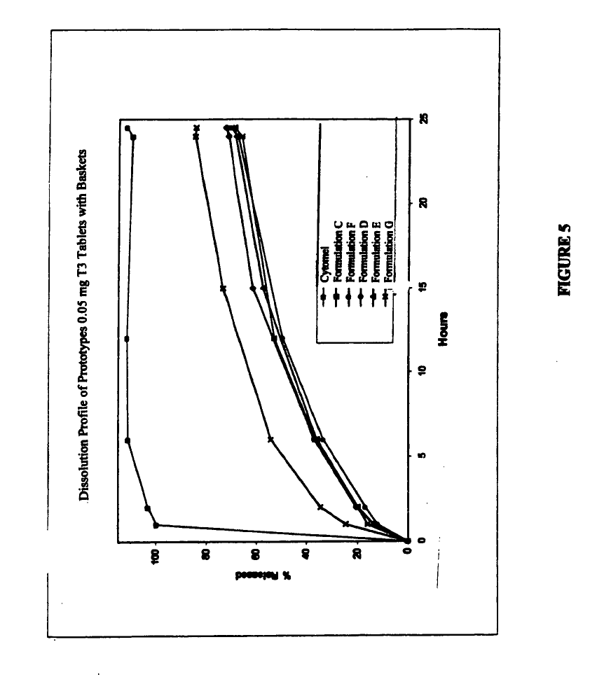

FIG. 4 shows dissolution profile information for tablets made from Formulation B.

FIG. 5 shows dissolution profile information for tablets made from Formulations C through G.

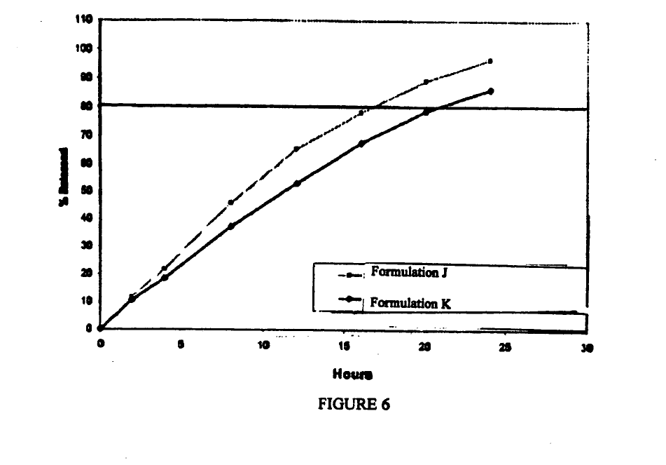

FIG. 6 shows dissolution profile information for tablets made from Formulations J and K.

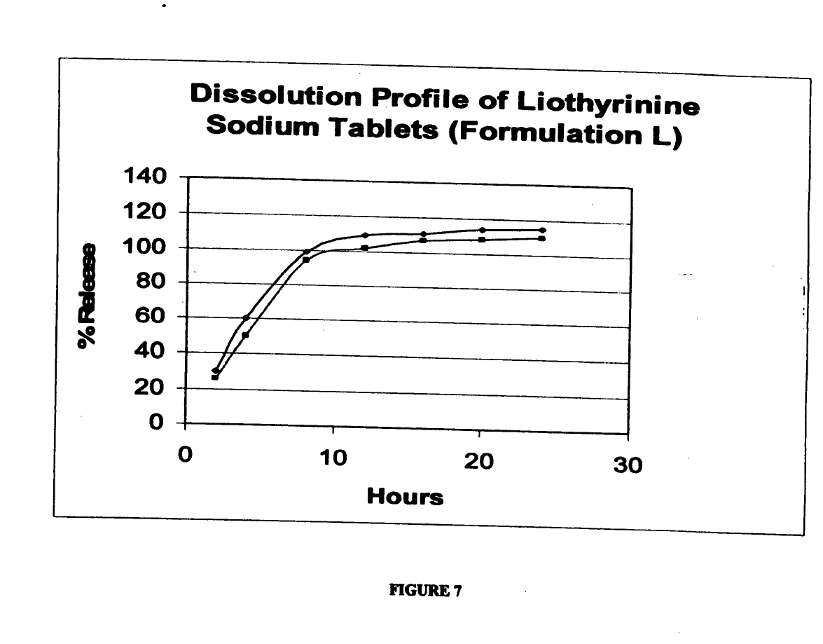

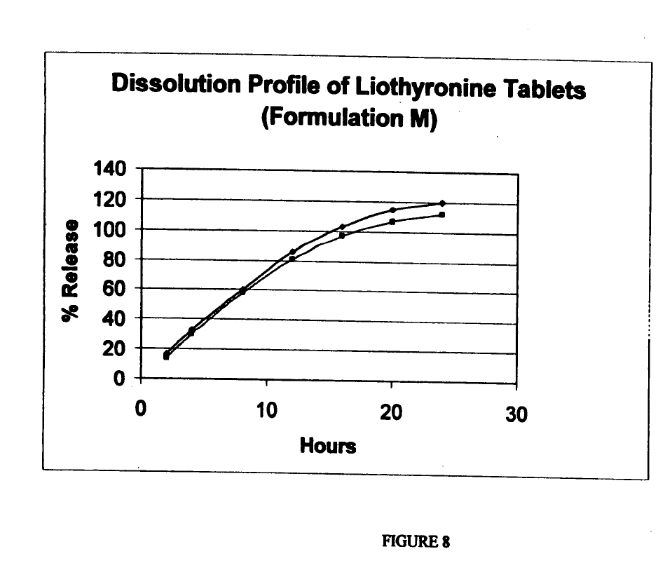

FIG. 7 shows dissolution profile information for tablets made from Formulation L.

FIG. 8 shows dissolution profile information for tablets made from Formulation M.

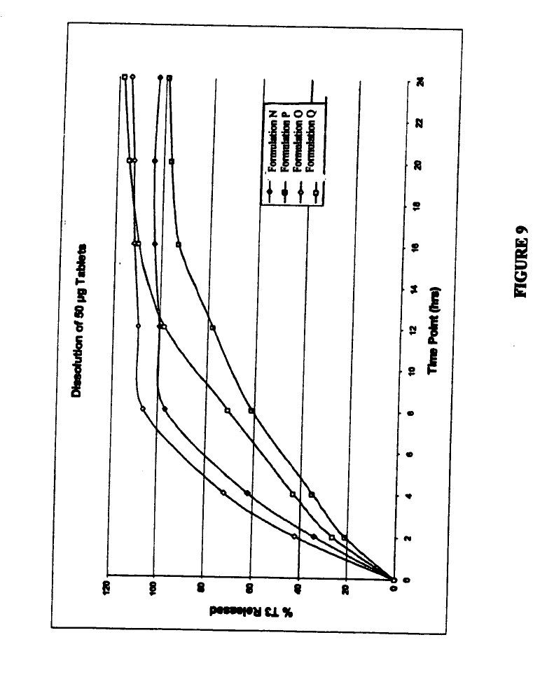

FIG. 9 shows dissolution profile information for tablets made from Formulations N through Q.

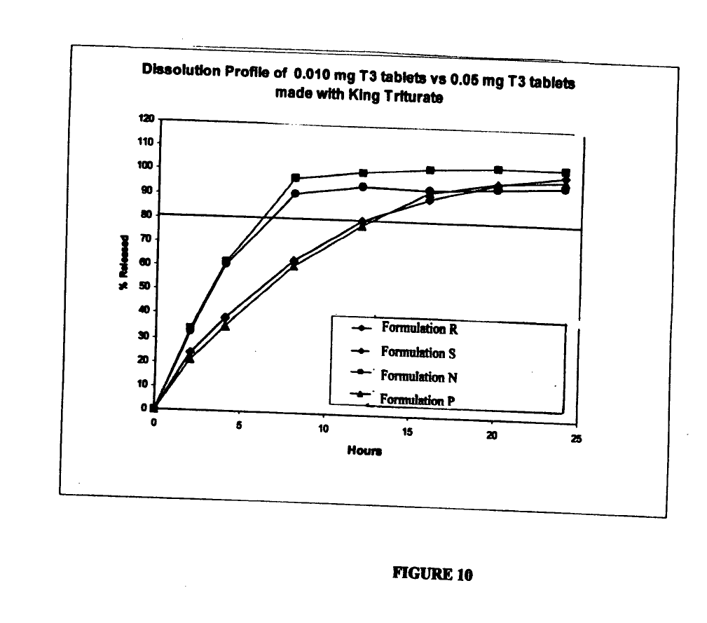

FIG. 10 shows dissolution profile information for tablets made from Formulations R and S.

### 5. DETAILED DESCRIPTION

5.1. Liothyronine

In certain embodiments the pharmaceutical compositions of the present invention include liothyronine or a pharmaceutically acceptable salt, prodrug or stereoisomer thereof. Liothyronine is the synthetic form of a natural hormone. The preferred form of liothyronine is liothyronine salt and, in the present invention, the preferred salt is liothyronine sodium.

Though the present invention encompasses sustained release pharmaceutical compositions of liothyronine and pharmaceutical salts thereof, the present invention is not limited to sustained release pharmaceutical compositions of liothyronine. The sustained release pharmaceutical compositions of the present invention can also be used in connection with other active pharmaceutical ingredients (“APIs”), such as other hormones (either natural or synthetic) and, in particular, other thyroid hormones. Examples of other thyroid hormones include, but are not limited to, L-thyroxine and triiodothyronine.

5.2. Compositions

The present invention is directed towards sustained release pharmaceutical compositions of liothyronine that eliminate, or at least mitigate, the initial liothyronine plasma concentration peak that is characteristic of currently available immediate release liothyronine formulations. In certain embodiments, prior to 1 hour after administration of the pharmaceutical compositions of the present invention, the plasma concentration of liothyronine does not exceed the baseline concentration of liothyronine by more than 3.5 times that of the baseline concentration. In other embodiments, prior to 1 hour after administration, the concentration of liothyronine does not exceed the baseline concentration of liothyronine by more than 3.0 times, or more than 2.5 times, or more than 2.0 times, or more than 1.5, or more than 1.0 times that of the baseline concentration.

In other embodiments, prior to 2 hours after administration of the pharmaceutical compositions of the present invention, the plasma concentration of liothyronine does not exceed the baseline concentration of liothyronine by more than 3.5 times that of the baseline concentration. In other embodiments, prior to 2 hours after administration, the concentration of liothyronine does not exceed the baseline concentration of liothyronine by more than 3.0 times, or more than 2.5 times, or more than 2.0 times, or more than 1.5, or more than 1.0 times that of the baseline concentration.

The sustained release pharmaceutical compositions of the present invention are also directed towards reducing fluctuations of liothyronine plasma concentrations during treatment as compared with currently available immediate release liothyronine formulations. Also, the controlled release pharmaceutical compositions of the present invention are designed to be able to allow a subject to achieve an optimal plasma level concentration of liothyronine and reduce or eliminate undesired plasma level fluctuations above or below the subject's optimal plasma level concentration of liothyronine. In most subjects the optimal plasma level concentration of liothyronine is 80-180 ng/dL. For example, in certain embodiments the pharmaceutical compositions of the present invention, prevent or reduce plasma level concentration fluctuations that exceed 80%, 75%, 70%, 65%, 60% or 55%, 50%, 45%, 40%, 35%, 30%, 35%, 20%, 25%, 20%, 15%, 10%, 5% of the optimal plasma level concentration of liothyronine.

In certain embodiments after 1 hour after administration of the pharmaceutical compositions of the present invention, the plasma concentration of liothyronine does not fluctuate more than 80%, 75%, 70%, 65%, 60% or 55% per hour. In other embodiments the plasma concentration of liothyronine does not fluctuate more than 50% per hour. For example, if 1 hour after administration of the pharmaceutical compositions of the present invention, the liothyronine concentration is 0.209 ng/ml, then the plasma concentration of liothyronine at 2 hours after administration will be between 0.104 ng/ml to 0.314 ng/ml. In other embodiments after 1 hour post administration of the pharmaceutical compositions of the present invention, the plasma concentration of liothyronine does not fluctuate more than 45%, 40%, 35%, 30%, 35%, 20%, 25%, 20%, 15%, 10%, 5%, 4%, 3%, 2% or 1% per hour.

Additionally, in some embodiments of the present invention, after 1 hour after administration of the pharmaceutical compositions of the present invention, the plasma concentration of liothyronine does not fluctuate more than ±5, 4.5, 4, 3.5, 3, 2.5, 2, 1.5, 1 or 0.5 ng/dL/hr. In other embodiments, the plasma concentration of liothyronine does not fluctuate more than ±50, 45, 40, 35, 30, 25, 20, 15, or 10 ng/dL/hr.

In certain embodiments the pharmaceutical compositions of the present invention can prevent or at least reduce plasma level concentrations that exceed 95%, 90%, 85%, 80%, 75%, 70%, 65%, 60%, 55%, 50%, 45%, 40%, 35%, 30%, 25%, 20%, 15%, 10%, or 5% of the baseline plasma level concentration of liothyronine.

Additionally, the sustained release compositions of the present invention are directed to compositions of liothyronine that are able to delay the Cₘₐₓ of liothyronine as compared to currently available immediate release formulations, as well as, compositions that can release a therapeutically effective amount of liothyronine for an extended period of time.

The Cₘₐₓ of liothyronine can occur at least 1, 2, 3, 4, 5, 6, 7, 8 hours or more after administration of the pharmaceutical compositions of the present invention. In certain embodiments the Cₘₐₓ of liothyronine occurs about 3 to 8 hours after administration of the pharmaceutical compositions of the present invention. In preferred embodiments of the present invention the Cₘₐₓ of liothyronine occurs about 3, 4 or 5 hours after administration of the pharmaceutical compositions of the present invention.

The sustained release compositions of the present invention are also directed to compositions of liothyronine that are able to prolong the Tₘₐₓ of liothyronine. Tₘₐₓ is the time at which Cₘₐₓ is achieved. The Tₘₐₓ of liothyronine can be greater than one hour post administration. In certain embodiments the Tₘₐₓ can be greater than 2, 3, 4, 5, 6, 7 or 8 hours post administration. In other embodiments the Tmax can be greater than 10, 12, 16, 24, 36 or 48 hours post administration of the controlled release compositions of the present invention. Ideally the Tₘₐₓ of liothyronine occurs between 6 to 12 hours post administration of the controlled release compositions of the present inventions. In some preferred embodiments the Tₘₐₓ of liothyronine occurs between 2 to 4 hours post administration of the controlled release composition of the present invention.

The controlled release pharmaceutical compositions comprising liothyronine, or a pharmaceutically acceptable salt thereof, release liothyronine so as to reduce the frequency or eliminate the occurrence of undesirable side effects. Such undesirable side effects include adverse cardiac effects. Such adverse cardiac effects include, but are not limited to, fluctuations in heart rate, fast or irregular heartbeat, heart palpitations, increased blood pressure, increased risk of heart attack, chest pain, and congestive heart failure. Other undesirable side effects may include headaches, skin rash or hives, confusion, mood swings, irritability, muscle weakness, psychosis, restlessness, nervousness, sweating, sensitivity to heat, anxiousness, excessive sweating, flushing, shortness of breath, osteoporosis and deceased bone density.

Upon administration of the controlled release compositions of the present invention such undesirable side effects can be reduced by about 10% or more, as compared to currently available immediate release formulations. In certain embodiments, undesirable side effects can be reduced by at least 10%, 15%, 20%, 25%, 30%, 35%, 40%, 45%, 50%, 55%, 60%, 65%, 70%, 75%, 80%, 85%, 90%, 95% or 100%, as compared to immediate release formulations of liothyronine.

For some side effects a reduction in the frequency or occurrence of undesirable side effects associated with immediate release formulations can be measured within the first hour post administration of the controlled release formulations of the present invention. As for other side effects a reduction in side effects can be measured within 24 or 48 hours, or longer post administration of the controlled release formulations of the present invention.

The reduction in frequency or elimination of the occurrence of undesirable side effects can be measured by any means known in the art. For example scales similar to the Crooks scale and the Klein Hyperthyroid Symptom Scale, which are used to measure hyperthyroidism symptoms, can be used to measure a reduction of the undesirable side effects associated with currently available immediate release liothyronine formulations. See Klein et al., Symptom Rating Scale for Assessing Hyperthyroidism, 148 Arch. Intern. Med. 387(1988). Also, side effects such as increased blood pressure and fluctuations in heart rate can be measured directly using methods known in the art.

The sustained release pharmaceutical compositions of the present invention can release a therapeutically effective amount of liothyronine for a period of at least 2 hours or longer. The pharmaceutical compositions of the present invention can release a therapeutically effective amount of liothyronine over a period of about 2 to 24 hours or longer. Additionally, a therapeutically effective amount of liothyronine can be released over a period of about 4 to 12 hours. Alternatively, the compositions of the present invention can release a therapeutically effective amount of liothyronine for at least a period of 8 to 12 hours. In certain embodiments the pharmaceutical compositions of the present invention release a therapeutically effective amount of liothyronine over a period of 2, 3, 4, 5, 6, 7, 8, 9, 10, 11, 12, 13, 14, 15, 16, 17, 18, 19, 20, 21, 22, 23, 24 hours or longer. Preferred pharmaceutical compositions release an effective amount of liothyronine over a period of 8, 12, 20 or 24 hours.

A therapeutically effective amount of liothyronine can be an amount of about 0.001 μg/kg/hour to about 100 μg/kg/hour or an amount of about 0.01 μg/kg/hour to about 10 μg/kg/hour or an amount of about 0.1 μg/kg/hour to about 1 μg/kg/hour.

Additionally, the release of liothyronine can follow zero-order or first order kinetics. Zero-order kinetics is attained by a constant rate of release of liothyronine, while first-order kinetics is attained by an initial fast release rate which is followed by a slower release rate.

The sustained release pharmaceutical compositions of the present invention can contain about 0.001% to about 10% of liothyronine by weight. Preferably, the compositions of the present invention contain about 0.01% to about 1% of liothyronine by weight. More preferably, the compositions of the present invention contain about 0.01 % to about 0.06% of liothyronine by weight.

The sustained release pharmaceutical compositions of the present invention can release 75%-90% of the liothyronine or a pharmaceutically acceptable salt thereof in about 8, 12, 15, 17, 19, 20, 22, or 24 hours or more. In certain embodiments, the sustained release pharmaceutical compositions of the present invention can release 80% of the liothyronine or a pharmaceutically acceptable salt thereof in about 8, 12, 15, 17, 19, 20, 22, or 24 hours. In other embodiments, the sustained release pharmaceutical compositions of the present invention can release 85% of the liothyronine or a pharmaceutically acceptable salt thereof in 24 hours or more.

In certain embodiments, the release rate of liothyronine from the sustained release pharmaceutical compositions, of the present invention, can be about 0.001 μg/hour to about 100 μg/hour of liothyronine. Additionally, the release rate of the sustained release pharmaceutical compositions, of the present invention, can be about 0.01 μg/hour to about 10 μg/hour, or about 0.1 μg/hour to about 10 μg/hour, or about 1 μg/hour to about 5 μg/hour.

The pharmaceutical compositions of the present invention may contain any therapeutically effective amount of liothyronine, such as from about 0.001 μg or less to about 200 μg or more, or preferably from about 0.01 μg to about 100 μg or preferably from about 0.1 μg to about 50 μg. Preferably, the dosage will be 5 μg, 10 μg, 25 μg or 50 μg. The pharmaceutical compositions of the present invention may contain any therapeutically effective amount of liothyronine, such as from about 0.001 μg/day or less to about 200 μg/day or more, or preferably from about 0.01 μg/day to about 100 μg/day or preferably from about 0.1 μg/day to about 50 μg/day. Preferably, the dosage will be 1 μg/day, 5 μg/day, 10 μg/day, 25 μg/day or 50 μg/day. In addition, the pharmaceutical compositions of the present invention can include pharmaceutically acceptable excipients, such as a polymer that can act as a rate limiting matrix.

5.3. Excipients

Pharmaceutical compositions of the present invention may also include a pharmaceutically acceptable excipient. Suitable pharmaceutically acceptable excipients include, but are not limited to, polymers, diluents, binders, glidants, vehicles, carriers, disintegrating agents, lubricants, swelling agents, solubilizing agents, wicking agents, cooling agents, preservatives, stabilizers, sweeteners, flavors, etc. While any pharmaceutically acceptable excipient is contemplated by the present invention, it should be understood that the excipient(s) selected for formulating with liothyronine should not defeat the controlled release objectives of the present invention.

Suitable polymers are able to form rate-limiting matrices that allow the liothyronine to be released in a controlled manner. In certain embodiments of the invention the controlled release of liothyronine is achieved with the aid of a hydrophilic polymer matrix. Hydrophilic polymers suitable for use in the present invention include, but are not limited to, water-soluble polymers, polymers soluble in intestine (enteric polymers), polymers soluble in stomach (stomach-soluble polymers), and polymers soluble in both stomach and intestine (stomach/intestine-soluble polymers).

Examples of suitable polymers include, but are not limited to, polysaccharides, celluloses, and organic moieties such as polyvinyl pyrrolidines and plastics.

Examples of celluloses include, but are not limited to, hydroxypropylcellulose, hydroxypropylmethylcellulose (a.k.a. hypromellose), hydroxyethylcellulose, ethylcellulose, cellulose acetate phthalate, cellulose acetate, hydroxypropyl methyl cellulose acetate succinate, hydroxypropyl methyl cellulose succinate, hydroxylpropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methylcellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butryrate trimellitate, cellulose acetate terephthalate, cellulose acetate isophthalate, cellulose acetate pyridine dicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid, cellulose acetate, ethyl picolinic acid cellulose acetate. These polymers can be used individually or in combination.

Other polymers that may be suitable for use with the present invention include, but are not limited to, acrylate and methacrylate copolymers. Exemplary commercial grades of such copolymers include the EUDRAGIT® series.

Other suitable polymers include, but are not limited to, proteins such as gelatin and albumin; starches such as carboxylic acid functionalized starches, starch glycolate, and cross-linked high amylose starch such as CONTRAMID®; carboxylic acid functionalized polymethyacrylates; carboxylic acid functionalized polyacrylate; amine-functionalized polyacrylates; amine-functionalized polymethacrylates; vinyl polymers and copolymers having at least one substituent selected from the group consisting of hydroxyl, alkylacyloxy, and cyclicamido; polyvinyl alcohols that have at least a portion of their repeat units in the unhydrolyzed (vinyl acetate) form; polyvinyl alcohol polyvinyl acetate copolymers; polyvinyl acetate phthalate; polyvinyl pyrrolidone; polyethylene polyvinyl alcohol copolymers, polyoxyethylene-polyoxypropylene copolymers, alkylacyloxy-containing repeat units, or cyclicamido-containing repeat units; polyvinyl alcohols that have at least a portion of their repeat units in the unhydrolyzed form; polyvinyl alcohol polyvinyl acetate copolymers; polyethylene glycol, polyethylene glycol polypropylene glycol copolymers, polyvinyl pyrrolidone polyethylene polyvinyl alcohol copolymers, and polyoxyethylene-polyoxypropylene block copolymers.

In certain embodiments the preferred polymer is hydroxypropylcellulose, hydroxypropyl methyl cellulose, hydroxymethyl cellulose, ethyl cellulose or a combination thereof.

The sustained release pharmaceutical compositions, of the present invention, can contain about 1% to about 99% of polymer by weight, or between 10% to about 90% of polymer by weight, or between 20% to about 80% of polymer by weight, or between 30% to about 70% of polymer by weight. Preferably, the compositions of the present invention contain about 40% or 60% of polymer by weight.

In certain embodiments of the present invention the pharmaceutical compositions can contain 5%, 10%, 15%, 20%, 25%, 30%, 35%, 40%, 45%, 50%, 55%, 60%, 65%, 70%, 75%, 80%, 85%, 90% or 95% of polymer by weight.

For example, the sustained release pharmaceutical compositions of the present invention can contain about 20% to about 80% of hydroxypropylmethylcellulose by weight. Preferably, the compositions of the present invention contain about 30% to about 70% of hydroxypropylmethylcellulose by weight. More preferably, the compositions of the present invention contain about 40% to 60% of hydroxypropylmethylcellulose by weight.

Examples of stabilizers or preservatives include, but are not limited to, parahydroxybenzoic acid alkyl esters, antioxidants, antifungal agents, and other stabilizers/preservatives known in the art.

Examples of coloring agents include, but are not limited to, water soluble dye, Aluminum Lake, ion oxide, natural colors, titanium oxide, and the like. Suitable Aluminum Lake coloring agents include, but are not limited to, FD&C Blue #1 Aluminum Lake, FD&C Red #30 Aluminum Lake, FD&C Red #40 Aluminum Lake, FD&C Yellow #6 Aluminum Lake, FD&C Yellow #10 Aluminum Lake or combinations thereof.

Examples of diluents or fillers include, but are not limited to, water-soluble and/or water-insoluble tabletting fillers. The water-soluble diluent agent may be constituted from a polyol of less than 13 carbon atoms, in the form of directly compressible material (the mean particle size being about 100 and about 500 microns), in the form of a powder (the mean particle size being less than about 100 microns) or a mixture thereof. The polyol is preferably chosen from the group comprising of mannitol, xylitol, sorbitol and maltitol. The water-insoluble filler maybe a cellulosic derivative, such as, microcrystalline cellulose or a starch, such as, pre-gelatinized starch. Preferred diluents are lactose monohydrate, microcrystalline cellulose, silicified microcrystalline cellulose, calcium sulfate and magnesium oxide.

Examples of disintegrating agents include, but are not limited to, cross-linked sodium carboxymethylcellulose, crospovidone and their mixtures.

Examples of lubricating agents include, but are not limited to, magnesium stearate, stearic acid and its pharmaceutically acceptable alkali metal salts, sodium stearyl fumarate, Macrogol 6000, glyceryl behenate, talc, colloidal silicon dioxide, calcium stearate, sodium stearate, Cab-O-Sil, Syloid, sodium lauryl sulfate, sodium chloride, magnesium lauryl sulfate, talc and mixtures thereof.

Examples of swelling agents include, but are not limited to, starches; polymers; cellulosic materials, such as, microcrystalline cellulose, hydroxypropylmethyl cellulose, sodium carboxymethylcellulose and ethyl cellulose; waxes such as bees wax; natural materials, such as, gums and gelatins; or mixtures of any of the above.

Examples of glidants include, but are not limited to, silicone dioxide.

A flavoring may be advantageously chosen to give a combination of fast onset and long-lasting sweet taste and get a “round feeling” in the mouth with different textures or additives. Cooling agents can also be added in order to improve the mouth feeling and provide a synergy with flavors and sweetness. Various other materials may be present as coatings or to otherwise modify the physical form of the dosage unit. For instance, tablets or capsules may be coated with shellac, sugar or both.

Preferred pharmaceutical compositions of the invention comprise liothyronine sodium, a polymer, a filler, a glidant and a lubricant. For example, one preferred pharmaceutical composition of the invention comprises liothyronine sodium, hydroxypropylmethylcellulose (e.g., Methocel®, Dow Chemical Corp., Midland, Mich.), microcrystalline cellulose, colloidal silicon dioxide and stearic acid. Other preferred pharmaceutical compositions of the invention comprise liothyronine sodium, a polymer, a filler, a glidant, a diluent and a lubricant. For example, one preferred pharmaceutical composition of the invention comprises liothyronine sodium, calcium sulfate hydroxypropyl methyl cellulose (e.g., Methocel®, Dow Chemical Corp., Midland, Mich.), microcrystalline cellulose, colloidal silicon dioxide and stearic acid.

5.4. Administration

Pharmaceutical compositions of the invention comprise liothyronine, or a pharmaceutically acceptable salt, prodrug or stereoisomer thereof, which can be effectively administered to patients orally. Oral pharmaceutical compositions of the present invention are generally in the form of individualized or multiunit doses, such as coated or uncoated tablets, caplets, powders, suspension tablets, chewable tablets, rapid melt tablets, capsules, e.g., a single or double shell gelatin capsule, tablet-filled capsules, effervescent powders, effervescent tablets, pellets, multi-particulates, granules, liquids, solutions, or suspensions, respectively.

While the present invention contemplates any solid pharmaceutical composition suitable for oral administration, liothyronine tablets, capsules, tablet-filled capsules and caplets are especially preferred. When the pharmaceutical compositions of the present invention are formed into tablets or caplets, it is to be understood that the tablets or caplets may be scored, and that they may be of any suitable shape and size, such as round, square, rectangular, oval, diamond, pentagon, hexagon or triangular, so long as the objectives of the present invention are not defeated. It is to be further understood that when tablet-filled capsules are selected, the tablets utilized therewith may be formed into shapes that either (a) correspond to the capsules to permit over-coating or encapsulation via the capsules or (b) readily fit inside the capsules. In certain embodiments of the present invention the pharmaceutical composition is a round, concave tablet.

The amount of surface area of the pharmaceutical dosage forms of the present invention can affect the release profile of liothyronine. In certain embodiments the surface area of the pharmaceutical dosage forms of the present invention can be from 1.0 to 0.01 in². More specifically, the surface area can be from 0.5 to 0.05 in². In preferred embodiments the dosage form is a round, concave tablet having a surface area of 0.2 to 0.1 in². In one embodiment the dosage form is a round, concave tablet having a surface area of 0.71 in².

The present invention also provides methods of using such pharmaceutical compositions for the prevention, treatment and management of various disease and conditions caused by deficient thyroid hormone, including, but not limited to, thyroid hormone deficiency and hypothyroidism.

Additionally, preferred pharmaceutical compositions of the present invention can be administered to a patient to treat or prevent congestive heart failure (CHF).

5.5. Methods of Making

The present invention also provides methods of making the pharmaceutical compositions described herein. Pharmaceutical compositions of the invention may be made by a variety of well-known techniques.

In certain methods of making pharmaceutical compositions encompassed by the invention, a desired amount of microcrystalline cellulose and liothyronine are measured-out, and passed through a screen, after which they are mixed using a pestle. After this, additional excipients are passed through a screen, placed in the first blender, and mixed (e.g., for about 1 minute). The microcrystalline cellulose and the liothyronine mixture is added to the blender, and the resulting combination is mixed for an additional period of time (e.g., about 5 minutes). An additional amount of microcrystalline cellulose is added to the blender and is mixed for an additional amount of time (e.g., about 5 more minutes). This last step is repeated an additional time.

In a second blender, equipped with an intensifier bar, half of the desired amount of hypromellose, the full amount of silicone dioxide and any additional microcrystalline cellulose are mixed (e.g., for about 5 minutes) with the intensifier bar off. The contents of the first blender are then transferred to the second blender and mixed (e.g., for about 5 minutes) with the intensifier bar off, after which the bar may be turned on and the mixture allowed to mix for an additional amount of time (e.g., about 30 minutes). After this, the remaining amount of hypromellose is added, and the mixture is allowed to mix (e.g., for about 5 minutes) with the intensifier bar off. The intensifier bar is then turned on, and the mixture is allowed to mix for an additional amount of time (e.g., about 30 minutes). The resulting mixture is then formed into tablets using conventional methods.

A flow chart of the above method of making the compositions of the present invention is shown in FIG. 1.

In other methods of making pharmaceutical compositions encompassed by the invention, first a triturate or powder blend of liothyronine salt and calcium sulfate is formed. To make the blend, calcium sulfate dihydrate is charged to a planetary mixer. Liothyronine sodium is charged into an indention in the calcium sulfate dihydrate. The ingredients are mixed in the planetary mixer. Once mixing is complete, the blend is passed through an air jet mill under nitrogen. Upon completion of the pulverization step, the blend is charged into a clean planetary mixer and blended.

Next a portion of microcrystalline cellulose is delumped, charged to a V-blender and mixed. Another portion of microcrystalline cellulose is delumped and charged into a mixing bowl. The liothyronine sodium triturate is charged into the mixing bowl (without delumping) and mixed with the microcrystalline cellulose until visually homogenous. Using a portion of the microcrystalline cellulose from the V-blender as the rinse, the triturate is transferred via rinsing from the mixing bowl to the V-blender and the mixture is blended. Another portion of microcrystalline cellulose is delumped, charged to the V-blender and mixed. An additional portion of microcrystalline cellulose is delumped, charged to the V-blender and mixed. The colloidal silicon dioxide is delumped using a portion of the microcrystalline cellulose for facilitation. A portion of the hypromellose is delumped and charged into another V-blender (larger) along with the remaining microcrystalline cellulose and blended. The contents of the first V-blender is transferred to the second (larger) V-blender and blended utilizing the intensifier bar. The final portion of the hypromellose is delumped and transferred to the (larger V-blender) and blended with the intensifier bar. A portion (˜20%) of the material is removed from the blender, stearic acid is delumped and added to the blender and the removed material replaced. The final blend is mixed without the intensifier bar until it is homogenous. Blend homogeneity is confirmed via 10 point analytical blend analyses, samples are properly labeled and provided to the laboratory, and the blend is discharged into a properly labeled suitable container with desiccant for transfer to tableting.

Tablets can be compressed to a weight of 100 mg (±5%) on a rotary tablet press with 0.2500″ round concave tooling (with embossing of “1” for Formulation 1 and embossing of “2” for Formulation 2). Tablets may be dedusted. The acceptable tablets can further be placed into properly labeled, suitable containers for transfer to packaging.

A flow chart of the above method of making the compositions of the present invention is shown in FIG. 2.

Since APIs (e.g., liothyronine) are sensitive to heat care should be taken to limit exposure to inappropriate amounts of various levels during pharmaceutical manufacturing process. Additionally, adding known stabilizers, such as Ceolus® KG-802 microcrystalline cellulose (Asahi Kasei), to the pharmaceutical formations can be of use. In addition, if the pharmaceutical composition is a tablet, the tabulating processes itself can be adjusted to avoid excessive pressures, which can also lead to API degradation. Examples of preferred tabulating conditions aim for tablet hardnesses of about 4 to about 5 kp (See, e.g., Examples, infra).

### EXAMPLES

Tablets with Formulation A through G

100 mg tablets containing 0.051 mg liothyronine sodium and 40% hydroxypropyl methyl cellulose were prepared from formulation shown in Table 1.

TABLE 1

| | mg/tablet | |
|---|---|---|
| Ingredient | Formulation A | Formulation B |
| Liothyronine Sodium | 0.051 | 0.051 |
| Microcrystalline Cellulose | 56.649 | 56.649 |
| (Ceolus ® KG-802) | | |
| Hypromellose 2208 (Methocel ® | 40.000 | 0 |
| K100LV Premium CR) | | |
| Hypromellose 2208 (Methocel ® | 0 | 40.000 |
| K4M Premium CR) | | |
| Colloidal Silicon Dioxide | 0.300 | 0.300 |
| (Cab-o-sil ® M-5) | | |
| Stearic Acid, NF | 3.000 | 3.000 |
| Total | 100 | 100 |

Tablets containing 0.0602 mg liothyronine sodium and 20% hydroxypropyl methyl cellulose were prepared from formulations shown in Table 2.

TABLE 2

| | Formulation C | | | Formulation D | | | Formulation E | | |
|---|---|---|---|---|---|---|---|---|---|
| Ingredient | Mg/Tablet | % Tablet | Mg/Batch | Mg/Tablet | % Tablet | Mg/Batch | Mg/Tablet | % Tablet | g/Batch |
| Liothyronine | 0.0602 | 0.0602 | 0.3010 | 0.0602 | 0.0602 | 0.3010 | 0.0602 | 0.0602 | 0.3010 |
| Sodium | | | | | | | | | |
| Ceolus ® | 66.9398 | 66.9398 | 334.6990 | 76.9398 | 76.9398 | 334.6990 | 66.9398 | 66.9398 | 334.6990 |
| KG-802 | | | | | | | | | |
| Methocel ® | 20.0000 | 20.0000 | 100.0000 | 10.0000 | 10.0000 | 100.0000 | 15.0000 | 15.0000 | 75.0000 |
| K15MP CR | | | | | | | | | |
| Methocel ® | 0 | 0 | 0 | 0 | 0 | 0 | 5.0000 | 5.0000 | 25.0000 |
| K100MP | | | | | | | | | |
| CR | | | | | | | | | |
| Avicel ® PH | 10.0000 | 10.0000 | 50.0000 | 10.0000 | 10.0000 | 50.0000 | 10.0000 | 10.0000 | 50.0000 |
| 101 | | | | | | | | | |
| Stearic Acid | 3.0000 | 3.0000 | 15.0000 | 3.0000 | 3.0000 | 15.0000 | 3.0000 | 3.0000 | 15.0000 |
| Total | 100.0000 | 100.0000 | 500.0000 | 100.0000 | 100.0000 | 500.0000 | 100.0000 | 100.0000 | 500.0000 |

Tablets containing 0.0602 mg liothyronine sodium and between 15% to 25% hydroxypropyl methyl cellulose were prepared from formulations shown in Table 3.

TABLE 3

| | Formulation F | | | Formulation G | | |
|---|---|---|---|---|---|---|
| Ingredient | Mg/Tablet | % Tablet | g/Batch | Mg/Tablet | % Tablet | g/Batch |
| Liothyronine | 0.0602 | 0.0602 | 0.3010 | 0.0602 | 0.0602 | 0.3010 |
| Sodium | | | | | | |
| Ceolus ® KG- | 71.9398 | 71.9398 | 359.6990 | 81.9398 | 81.9398 | 409.6990 |
| 802 | | | | | | |
| Methocel ® | 25.0000 | 25.0000 | 125.0000 | 15.0000 | 15.0000 | 75.0000 |
| K15MP CR | | | | | | |
| Methocel ® | 0 | 0 | 0 | 0 | 0 | 0 |
| K100MP CR | | | | | | |
| Stearic Acid | 3.0000 | 3.0000 | 15.0000 | 3.0000 | 3.0000 | 15.0000 |
| Total | 100.0000 | 100.0000 | 500.000 | 100.0000 | 100.0000 | 500.0000 |

Manufacture of Tablets of Formulation A through G

Tablets were made from the ingredients described above in Table 1 through Table 3 the following steps:

1. Weigh out 100 g of Ceolus® KG-802 and pass through a #40-mesh hand screen, followed by the Liothyronine Sodium, collect in an appropriate container and mix using a pestle.

2. Screen all remaining excipients through the #40-mesh hand screen.

3. Charge 100 g pre-screened Ceolus® KG-802 into a 4 qt PK blender and mix for 1 minute.

4. Charge Step 1 Ceolus/API mixture to the blender and mix for 5 minutes.

5. Rinse the API/Ceolus container with 200 g pre-screened Ceolus® KG-802, charge to the 4 qt PK blender and mix for 5 minutes.

6. Charge 400 g pre-screened Ceolus® KG-802 to the blender and mix for 5 minutes.

7. To a 16 qt. PK blender equipped with an intensifier bar charge ½ of the pre-screened Methocel®, all of the Cab-o-sil® and remaining prescreened Ceolus® KG-802 and mix for 5 minutes with intensifier bar off.

8. Transfer the content from the 4 qt. PK blender, Step 6, to the 16 qt. PK blender and mix for 15 minutes with the intensifier bar on. Take a 3-point blend uniformity sample and label blend samples as “Initial-15”.

9. Mix for an additional 15 minutes with the intensifier bar on and take a 3-point blend uniformity sample. Label blend samples as “Initial-30”.

10. Charge the remaining ½ pre-screened Methocel® to the 16 qt. PK blender and mix for 15 minutes with the intensifier bar on. Take a 3-point blend uniformity sample and label blend samples as “Intermediate-15”.

11. Mix for an additional 15 minutes with the intensifier bar on and take a 3-point blend uniformity sample. Label blend samples as “Intermediate-30”.

12. Charge Stearic Acid to the 16 qt. PK blender and mix for 5 minutes with the intensifier bar off. Take a 3-point blend uniformity sample and label blend samples as “Final-5”.

Tablets are prepared from the final material using a Korsch® PH 103 tablet press equipped with 0.2500″ round, standard concave, plain tooling at a press speed of about 36 RPM. The target weight was 100.0 mg±5% with a target hardness of 5 kp.

Tablets with Formulation H Through K

Tablets containing 0.051 μg -0.052 μg of liothyronine sodium and 40% to 60% hydroxypropyl methyl cellulose were prepared from formulations shown in Table 4.

TABLE 4

| | mg/tablet | | | |
|---|---|---|---|---|
| Ingredient | Formulation H | Formulation I | Formulation J | Formulation K |
| Liothyronine Sodium | 0.052 | 0.051 | 0.051 | 0.051 |
| Microcrystalline Cellulose | 56.649 | 56.649 | 56.649 | 36.649 |
| (Ceolus ® KG-802) | | | | |
| Hypromellose 2208 | 40.000 | 0 | 40.000 | 60.000 |
| (Methocel ® K100M | | | | |
| Premium CR) | | | | |
| Hypromellose 2208 | 0 | 40.000 | 0 | 0 |
| (Methocel ® K4M | | | | |
| Premium CR) | | | | |
| Colloidal Silicon Dioxide | 0.300 | 0.300 | 0.300 | 0.3000 |
| (Cab-o-sil ® M-5) | | | | |
| Stearic Acid, NF | 3.000 | 3.000 | 3.000 | 3.000 |
| Total | 100 | 100 | 100 | 100 |

Manufacture of Tablets of Formulations H Through K

Tablets were made from the ingredients described above in Table 4 by the following steps:

1. Weigh out 25 g of Ceolus® KG-802 and pass through a #40-mesh hand screen, followed by the Liothyronine Sodium, collect in an appropriate container and mix using a pestle.

2. Screen all remaining excipients through the #40-mesh hand screen.

3. Charge 25 g pre-screened Ceolus® KG-802 into a 4 qt. PK blender and mix for 1 minute.

4. Charge Step 1 Ceolus/API mixture to the blender and mix for 5 minutes.

5. Rinse the API/Ceolus container with 50 g pre-screened Ceolus® KG-802, charge to the 2 qt. PK blender and mix for 5 minutes.

6. Charge 100 g pre-screened Ceolus® KG-802 to the blender and mix for 5 minutes.

7. To a 4 qt. PK blender equipped with an intensifier bar charge ½ of the pre-screened Methocel®, all of the Cab-o-sil® and remaining prescreened Ceolus® KG-802 and mix for 5 minutes with intensifier bar off.

8. Transfer the content from the 2 qt. PK blender, Step 6, to the 4 qt. PK blender and mix for 5 minutes with the intensifier bar off followed by 30 minutes with the intensifier bar on.

9. Charge the remaining ½ pre-screened Methocel® to the 4 qt. PK blender and mix for 5 minutes with the intensifier bar off followed by 30 minutes with the intensifier bar on.

10. Pass Stearic Acid through a #60-mesh hand screen, charge to the 4 qt. PK blender and mix for 5 minutes with the intensifier bar off.

11. Pass the entire blend through a Comil® equipped with a 018R screen.

12. Place milled blend back in the 4 qt. PK blender and mix for 1 minute with the intensifier bar off.

Tablets are prepared from the final material using a Korsch® PH 103 tablet press equipped with 0.2500″ round, standard concave, plain tooling at a press speed of about 36 RPM. The target weight was 100.0 mg±5% with a target hardness of 5 kp.

Tablets with Formulation L and M

Additional tablets containing 0.0595 μg of liothyronine sodium and 40% hydroxypropyl methyl cellulose were prepared from formulations shown in Table 5.

TABLE 5

| | Formulation L | | Formulation M | |
|---|---|---|---|---|
| | 8 Hour Controlled | | 12 Hour Controlled | |
| | Release | | Release | |
| Ingredient | Mg/Tablet | g/Batch | Mg/Tablet | g/Batch |
| Liothyronine Sodium | 0.0595 | 2.9750 | 0.0595 | 2.9750 |
| Microcrystalline | 56.6405 | 2832.0250 | 56.6405 | 2832.0250 |
| Cellulose | | | | |
| (Ceolus ® KG-802) | | | | |
| Hypromellose 2208 | 40.0000 | 2000.0000 | 0 | 0 |
| (Methocel ® K100LV | | | | |
| Premium CR) | | | | |
| Hypromellose 2208 | 0 | 0 | 40.0000 | 2000.0000 |
| (Methocel ® K4M | | | | |
| Premium CR) | | | | |
| Colloidal Silicon Dioxide | .3000 | 15.0000 | .3000 | 15.0000 |
| (Cab-o-sil ® M-5) | | | | |
| Stearic Acid, NF | 3.0000 | 150.0000 | 3.0000 | 150.0000 |
| Total | 100.0000 | 5000.0000 | 100.0000 | 5000.0000 |

Manufacture of Formulations L and M

Tablets were made from the ingredients described above in Table 5 by the following steps:

1. Liothyronine was blended with the some of the Ceolus® in a geometric fashion in a 4 qt. PK blender.

2. A 16 qt. PK blender, equipped with an intensifier bar, was charged with half of the Methocel®, the Cab-o-sil® and the remaining Ceolus®.

3. The liothyronine and Ceolus® of Step 1 was added to the 16 qt. PK blender.

4. The mixture was stirred for 30-40 minutes.

5. The remaining Methocel was added and the mixture was stirred for an additional 30-40 minutes.

6. Stearic acid was added and the mixture was blended to form the final blend.

7. The blend is then tableted into a 100 mg total weight tablet using 0.2500″ round, standard concave tooling.

Tablets with Formulation N through W

50 μg tablets liothyronine sodium and 40% hydroxypropyl methyl cellulose were prepared from formulations shown in Table 6.

TABLE 6

| | g/batch | | | |
|---|---|---|---|---|
| | Formulation N | Formulation O | Formulation P | Formulation Q |
| Ingredient | (non-cGMP) | (cGMP) | (non-cGMP) | (cGMP) |
| Liothyronine Sodium | 2.5 | 2.6^{1,2,3} | 2.5 | 2.6^{1,2,3} |
| USP^{1,2} | | | | |
| Calcium Sulfate | 28.9² | 30.1^{2,3} | 28.9² | 30.1^{2,3} |
| Dihydrate NF² | | | | |
| Microcrystalline | 2803.6⁴ | 2802.3⁴ | 2803.6⁴ | 2802.3⁴ |
| Cellulose NF | | | | |
| (Ceolus ® KG-802) | | | | |
| Hypromellose USP | 0 | 0 | 2000 | 2000 |
| Type 2208 | | | | |
| (Methocel ® K4M | | | | |
| Premium CR) | | | | |
| Hypromellose USP | 2000 | 2000 | 0 | 0 |
| Type 2208 | | | | |
| (Methocel ® K100LV | | | | |
| Premium CR) | | | | |
| Colloidal Silicon | 15.0 | 15.0 | 15.0 | 15.0 |
| Dioxide NF | | | | |
| (Cab-o-sil ® M-5P) | | | | |
| Stearic Acid NF | 150 | 150 | 150 | 150 |
| Total Batch Weight | 5000.0⁵ | 5000.0⁵ | 5000.0⁵ | 5000.0⁵ |

¹2.6 g liothyronine sodium = 2.5 g liothyronine. Tablets formulated to deliver 50 μg of liothyronine.
²As Liothyronine Sodium Triturate containing 7.94% liothyronine sodium blended with calcium sulfate dihydrate.
³Correct for potency, moisture and overage (2%) to account for manufacturing losses.
⁴Adjust based on amount of active charged to batch.
⁵Batch size theoretically produces 50,000 tablets.

25 μg tablets liothyronine sodium and 40% hydroxypropyl methyl cellulose were prepared from formulations shown in Table 7.

TABLE 7

| | Formulation R | | | Formulation S | | |
|---|---|---|---|---|---|---|
| Ingredient | Mg/Tablet | % Tablet | g/Batch | Mg/Tablet | % Tablet | g/Batch |
| Liothyronine | 0.0259 | 0.0259 | 0.2590 | 0.0259 | 0.0259 | 0.2590 |
| Sodium | | | | | | |
| Calcium | 0.3003 | 0.3003 | 3.0030 | 0.3003 | 0.3003 | 3.0030 |
| Sulfate | | | | | | |
| Ceolus ® | 56.3738 | 56.3738 | 563.7380 | 56.3738 | 56.3738 | 563.7380 |
| KG-802 | | | | | | |
| Methocel ® | 40.0000 | 40.0000 | 400.0000 | 0 | 0 | 0 |
| K100LV | | | | | | |
| Premium CR | | | | | | |
| Methocel ® K4M | 0 | 0 | 0 | 40.0000 | 40.0000 | 400.0000 |
| Premium CR | | | | | | |
| Cab-o-Sil ® | 0.3000 | 0.3000 | 3.0000 | 0.3000 | 0.3000 | 3.0000 |
| Stearic Acid | 3.0000 | 3.0000 | 30.0000 | 3.0000 | 3.0000 | 30.0000 |
| Total | 100.0000 | 100.0000 | 1000.0000 | 100.0000 | 100.0000 | 1000.0000 |

10 μg tablets liothyronine sodium and 40% hydroxypropyl methyl cellulose were prepared from formulations shown in Table 8.

TABLE 8

| | Formulation T | | | Formulation U | | |
|---|---|---|---|---|---|---|
| | 10 μg Tablet | | | 10 μg Tablet | | |
| Ingredient | Mg/Tablet | % Tablet | g/Batch | Mg/Tablet | % Tablet | g/Batch |
| Liothyronine | 0.01036 | 0.01036 | 0.1036 | 0.01036 | 0.01036 | 0.1036 |
| Sodium | | | | | | |
| Calcium | 0.1200 | 0.1200 | 1.2000 | 0.1200 | 0.1200 | 1.2000 |
| Sulfate | | | | | | |
| Ceolus ® | 56.5696 | 56.5696 | 565.6964 | 56.5696 | 56.5696 | 565.6964 |
| KG-802 | | | | | | |
| Methocel ® | 40.0000 | 40.0000 | 400.0000 | 0 | 0 | 0 |
| K100LV | | | | | | |
| Premium CR | | | | | | |
| Methocel ® K4M | 0 | 0 | 0 | 40.0000 | 40.0000 | 400.0000 |
| Premium CR | | | | | | |
| Cab-o-Sil ® | 0.3000 | 0.3000 | 3.0000 | 0.3000 | 0.3000 | 3.0000 |
| Stearic Acid | 3.0000 | 3.0000 | 30.0000 | 3.0000 | 3.0000 | 30.0000 |
| Total | 100.0000 | 100.0000 | 1000.0000 | 100.0000 | 100.0000 | 1000.0000 |

5 μg tablets liothyronine sodium and 40% hydroxypropyl methyl cellulose were prepared from formulations shown in Table 9.

TABLE 9

| | Formulation V | | | Formulation W | | |
|---|---|---|---|---|---|---|
| | 5 μg Tablet | | | 5 μg Tablet | | |
| Ingredient | Mg/Tablet | % Tablet | g/Batch | Mg/Tablet | % Tablet | g/Batch |
| Liothyronine | 0.00518 | 0.00518 | 0.0518 | 0.00518 | 0.00518 | 0.0518 |
| Sodium | | | | | | |
| Calcium | 0.0600 | 0.0600 | 0.6000 | 0.0600 | 0.0600 | 0.6000 |
| Sulfate | | | | | | |
| Ceolus ® | 56.6348 | 56.6348 | 566.3482 | 56.6348 | 56.6348 | 566.3482 |
| KG-802 | | | | | | |
| Methocel ® | 40.0000 | 40.0000 | 400.0000 | 0 | 0 | 0 |
| K100LV | | | | | | |
| Premium CR | | | | | | |
| Methocel ® K4M | 0 | 0 | 0 | 40.0000 | 40.0000 | 400.0000 |
| Premium CR | | | | | | |
| Cab-o-Sil ® | 0.3000 | 0.3000 | 3.0000 | 0.3000 | 0.3000 | 3.0000 |
| Stearic Acid | 3.0000 | 3.0000 | 30.0000 | 3.0000 | 3.0000 | 30.0000 |
| Total | 100.0000 | 100.0000 | 1000.0000 | 100.0000 | 100.0000 | 1000.0000 |

Manufacture of Formulations N through W

Tablets were made from the ingredients described above in Table 6 through Table 9 by the following steps:

1. A liothyronine sodium triturate is made by first charging calcium sulfate dihydrate to a planetary mixer. Liothyronine sodium is then charged into an indention (made by hand) in the calcium sulfate dihydrate (dry rinsing of the bag containing the liothyronine sodium may be done utilizing a scoop of the calcium sulfate dihydrate removed from the mixing bowl). The ingredients are mixed in the planetary mixer. Once mixing is complete, the blend is passed through an air jet mill under nitrogen. Upon completion of the pulverization step, the blend is charged into a clean planetary mixer and blended.

2. A portion of microcrystalline cellulose is delumped, charged to a V-blender and mixed.

3. Another portion of microcrystalline cellulose is delumped and charged into a mixing bowl

4. The liothyronine sodium triturate is charged into the mixing bowl (without delumping) and mixed with the microcrystalline cellulose until visually homogenous. Using a portion of the microcrystalline cellulose from the V-blender as the rinse, the triturate mixture is transferred via rinsing from the mixing bowl to the V-blender. Rinses may be repeated as needed, and then the mixture is blended.

5. Another portion of microcrystalline cellulose is delumped, charged to the V-blender and mixed.

6. An additional portion of microcrystalline cellulose is delumped, charged to the V-blender and mixed.

7. The colloidal silicon dioxide is delumped using a portion of the microcrystalline cellulose for facilitation.

8. A portion of the hypromellose type 2208 is delumped and charged into another V-blender (larger) along with the remaining microcrystalline cellulose and blended.

9. The contents of the first V-blender is transferred to the second (larger) V-blender and blended utilizing the intensifier bar.

10. The final portion of the hypromellose is delumped and transferred to the (larger V-blender) and blended with the intensifier bar.

11. A portion (˜20%) of the material is removed from the blender, stearic acid is delumped and added to the blender and the removed material replaced.

12. The final blend is mixed without the intensifier bar until it is homogenous.

Blend homogeneity is confirmed via 10 point analytical blend analyses, samples are properly labeled and provided to the laboratory, and the blend is discharged into a properly labeled suitable container with desiccant for transfer to tableting. Tablets are compressed to a weight of 100 mg (±5%) on a rotary tablet press with 0.2500″ round concave tooling (for example with embossing of “N” for Formulation 1 and embossing of “M” for Formulation 2). Tablets may be dedusted. The acceptable tablets are placed into properly labeled, suitable containers for transfer to packaging.

Tablet Dissolution Profiles

Because of the buoyancy of tablets prepared according to Tables 1, 2 and 3, the use of wire “sinker cages,” or “wire sinkers,” were used to determine the dissolution rates of tablets of the invention. In this method, each tablet was placed in a 10 mesh, wire sinker cage, which was dropped in the dissolution medium (acetate buffer, pH 4.5) and submitted to USP Apparatus-2, paddle stirring element, at 100 RPM and tested for API (active pharmaceutical ingredient) concentration at interval time periods using high performance liquid chromatography.

Using this method, the dissolution profile of tablets of Formulation A are shown in FIG. 3; the dissolution profile of tablets of Formulation B are shown in FIG. 4; the dissolution profile of tablets of Formulations C through G are shown in FIG. 5; the dissolution profile of tablets of Formulations J and K are shown in FIG. 6; the dissolution profile of tablets of Formulation L are shown in FIG. 7; the dissolution profile of tablets of Formulation M are shown in FIG. 8; the dissolution profile of tablets of Formulations N through Q are shown in FIG. 9; and a comparison of the dissolution profile of 50 μg tablets of Formulations N and P and 25 μg tablets of Formulations R and S is shown in FIG. 10.

Stability Tests

Several 60 cc HDPE bottles with child-resistant screw caps and induction seal liners, containing 100 tablets and 1.0 g silica gel minipak dessicator and 6-8 inches of 12 g low moisture polyester coil. The tablets were then tested for stability at conditions which included 25° C./60% RH, 40° C./75% RH and 30° C./65% RH (storage only). The results of those tests are shown in Table 10.

TABLE 10

| Stability | | Results | | | | | | | | | | | | | |
|---|---|---|---|---|---|---|---|---|---|---|---|---|---|---|---|
| Test | Time Point | 00953-047 | | | | | | | 00953-051 | | | | | | |
| Appearance | Initial | Unchanged | | | | | | | Unchanged | | | | | | |
| | 1 month 25° C./60% RH | Unchanged | | | | | | | Unchanged | | | | | | |
| | 2 months 25° C./60% RH | Unchanged | | | | | | | Unchanged | | | | | | |
| | 3 months 25° C./60% RH | Unchanged | | | | | | | Unchanged | | | | | | |
| | 6 months 25° C./60% RH | Unchanged | | | | | | | Unchanged | | | | | | |
| | 1 month 40° C./75% RH | Unchanged | | | | | | | Unchanged | | | | | | |
| | 2 months 40° C./75% RH | Unchanged | | | | | | | Unchanged | | | | | | |
| | 3 months 40° C./75% RH | Unchanged | | | | | | | Unchanged | | | | | | |
| | 6 months 40° C./75% RH | Unchanged | | | | | | | Unchanged | | | | | | |
| | Hours | 2 | 4 | 8 | 12 | 16 | 20 | 24 | 2 | 4 | 8 | 12 | 16 | 20 | 24 |
| Dissolution | Initial | 20 | 41 | 82 | 96 | 102 | 104 | 107 | 13 | 27 | 54 | 76 | 91 | 102 | 107 |
| [n = 6] | 1 month 25° C./60% RH | 22 | 43 | 81 | 96 | 105 | 107 | 109 | 16 | 29 | 55 | 79 | 91 | 100 | 106 |
| (% Dissolved) | 2 months 25° C./60% RH | 22 | 42 | 80 | 93 | 99 | 104 | 106 | 12 | 26 | 54 | 78 | 97 | 105 | 109 |
| | 3 months 25° C./60% RH | 20 | 40 | 76 | 88 | 94 | 96 | 99 | 12 | 24 | 50 | 73 | 86 | 95 | 99 |
| | 6 months 25° C./60% RH | 19 | 40 | 74 | 88 | 93 | 96 | 98 | 11 | 22 | 48 | 71 | 88 | 97 | 103 |
| | 1 month 40° C./75% RH | 21 | 41 | 77 | 92 | 99 | 101 | 106 | 11 | 25 | 48 | 76 | 94 | 101 | 105 |
| | 2 months 40° C./75% RH | 19 | 40 | 78 | 87 | 93 | 94 | 98 | 11 | 24 | 50 | 72 | 86 | 93 | 96 |
| | 3 months 40° C./75% RH | 17 | 35 | 72 | 83 | 88 | 89 | 92 | 10 | 20 | 44 | 65 | 79 | 86 | 91 |
| | 6 months 40° C./75% RH | 15 | 33 | 68 | 81 | 86 | 90 | 93 | 8 | 19 | 43 | 66 | 80 | 90 | 95 |

| | | | |
|---|---|---|---|
| Assay | Initial | 98.9 | 96.8 |
| (% LC) | 1 month 25° C./60% RH | 93.3 | 94.3 |
| | 2 months 25° C./60% RH | 96.1 | 94.6 |
| | 3 months 25° C./60% RH | 93.2 | 95.9 |
| | 6 months 25° C./60% RH | 90.5 | 92.1 |
| | 1 month 40° C./75% RH | 95.0 | 93.8 |
| | 2 months 40° C./75% RH | 91.8 | 92.5 |
| | 3 months 40° C./75% RH | 90.4 | 91.1 |
| | 6 months 40° C./75% RH | 83.8 | 84.3 |
| Related | Initial | 0.5 | 0.5 |
| Substances | 1 month 25° C./60% RH | 0.7 | 1.0 |
| (% LC) | 2 months 25° C./60% RH | 0.8 | 1.1 |
| | 3 months 25° C./60% RH | 1.5 | 2.2 |
| | 6 months 25° C./60% RH | 1.6 | 2.5 |
| | 1 month 40° C./75% RH | 1.3 | 1.3 |
| | 2 months 40° C./75% RH | 1.5 | 2.1 |
| | 3 months 40° C./75% RH | 2.6 | 3.6 |
| | 6 months 40° C./75% RH | 3.4 | 3.6 |
| Hardness | Initial | 4.8, 3.0-6.5 | 5.0, 2.7-7.2 |
| [n = 10, | 1 month 25° C./60% RH | 5.4, 2.6-10.4 | 4.5, 2.7-6.8 |
| Range] (kp) | 2 months 25° C./60% RH | 4.7, 2.9-9.5 | 5.3, 3.3-7.5 |
| | 3 months 25° C./60% RH | 4.3, 3.3-5.4 | 5.1, 2.9-6.6 |
| | 6 months 25° C./60% RH | 4.6, 3.5-5.7 | 4.3, 2.8-6.2 |
| | 1 month 40° C./75% RH | 4.8, 3.0-5.7 | 5.4, 2.9-6.7 |
| | 2 months 40° C./75% RH | 4.3, 1.4-5.8 | 4.4, 2.8-6.5 |
| | 3 months 40° C./75% RH | 4.0, 1.7-6.7 | 4.9, 3.4-6.1 |
| | 6 months 40° C./75% RH | 4.3, 3.5-6.3 | 5.1, 3.1-6.0 |
| Karl Fischer | Initial | 4.7 | 4.0 |
| [n = 2] | 1 month 25° C./60% RH | 5.2 | 5.1 |
| (% Moisture) | 2 months 25° C./60% RH | 4.5 | 4.4 |
| | 3 months 25° C./60% RH | 5.0 | 4.9 |
| | 6 months 25° C./60% RH | 4.0 | 4.5 |
| | 1 month 40° C./75% RH | 5.2 | 4.9 |
| | 2 months 40° C./75% RH | 4.5 | 4.5 |
| | 3 months 40° C./75% RH | 5.0 | 4.9 |
| | 6 months 40° C./75% RH | 4.5 | 4.3 |

Additionally, stability tests were performed on 50 μg tablets of Formulations I through L. The stability results of tablets of Formulation N and Formulation P are shown in Table 11.

TABLE 11
50 μg Tablets Stability

| | Formula I | Formula J | Formula K | Formula L |
|---|---|---|---|---|
| Test | Initial | Initial | Initial | Initial |
| Assay % LC | 94.3 | 98.0 | 92.7 | 103.5 |
| 3,5 D-L-Tyrosine | 0.23% | ND | ND | ND |
| 3,5 D-L-Thyronine | 0.14% | 0.10% | 0.15% | 0.10% |
| 3,3,5 T-L-Thyronine | ND | ND | ND | ND |
| Levothryoxine | 0.37% | 0.45% | 0.43% | 0.54% |
| 3,3,5-T-thyroacetic Acid | 0.10% | ND | 0.14% | ND |
| 3,3,5,5-T-thyroacetic | ND | 0.13% | ND | 0.13% |
| Acid | | | | |
| Unknowns | ND | ND | ND | ND |
| Rel Subs (Total) | 0.8% | 0.7% | 0.7% | 0.8% |
| Water Content (n = 2) | 3.3% | 4.1% | 3.3% | 4.2% |
| Hardness (n = 10) | 6.0 kp | 5.2 kp | 5.5 kp | 5.9 kp |
| Dissolution (n = 6) | | | | |
| 2 hrs (NMT 50%) | 34 | 42 | 21 | 26 |
| 4 hrs | 62 | 72 | 35 | 43 |
| 8 hrs (NLT 60%) | 97 | 106 | 61 | 71 |
| 12 hrs (NLT 75%) | 100 | 109 | 78 | 98 |
| 16 hrs | 103 | 112 | 93 | 110 |
| 20 hrs | 104 | 113 | 97 | 115 |
| 24 hrs | 103 | 115 | 99 | 118 |

In-Vivo Tests of Tablets of Formulation H, I and K

Studies were done to determine the blood concentration of liothyronine (T3) in dogs. In this study, three groups of four male beagle dogs were each given a tablet prepared in accordance with the present invention, as shown in Table 5. Group 1 was given an 8-hour controlled release tablet (Formulation H). Group 2 was given a 12-hour controlled release tablet (Formulation I) and Group 3 was given a 20-hour controlled release tablet (Formulation K). All tablets, administered to the dogs, were oral tablets each containing 50 μg of liothyronine.

TABLE 12

| Group No. | No. of Animals | Formulation | Route | Dose (μg) |
|---|---|---|---|---|
| 1 | 4 | 1 Tablet | Oral | 50 |
| | | (Formulation H) | | |
| 2 | 4 | 1 Tablet | Oral | 50 |
| | | (Formulation I) | | |
| 3 | 4 | 1 Tablet | Oral | 50 |
| | | (Formulation K) | | |

Blood samples were taken prior to the administration of any tablets in order to establish a baseline. Then blood samples were taken at 1, 2, 4, 6, 8, 12, 16, 24, 30, 36, and 48 hours. The collection times of the dogs are shown in Table 13.

TABLE 13
Actual Versus Nominal Blood Sample Collection Times for Dogs Given Single Oral 50 μg
Doses of Liothyronine Formulated in 8-Hour, 12-Hour or 20-Hour Controlled Release Tablet

| | Group No. | Time Point (h) | | | | | | | | | | |
|---|---|---|---|---|---|---|---|---|---|---|---|---|
| Dog Number | Formulation | 1 | 2 | 4 | 6 | 8 | 12 | 16 | 24 | 30 | 36 | 48 |
| 1 | Group 1 | 0:59:49 | 1:59:48 | 3:59:55 | 5:59:50 | 7:59:50 | 11:59:30 | 15:59:23 | 24:00:37 | 30:00:18 | 35:59:30 | 48:00:35 |
| 2 | Lot no. | 0:59:48 | 1:59:53 | 3:59:32 | 5:59:46 | 7:59:37 | 11:59:28 | 15:59:47 | 23:59:44 | 29:59:44 | 35:59:49 | 48:00:15 |
| 3 | 00953-047 | 0:59:52 | 1:59:46 | 4:00:31 | 5:59:42 | 8:01:25 | 12:00:31 | 16:00:34 | 24:02:22 | 29:59:26 | 36:00:20 | 48:00:43 |
| 4 | 8-h | 1:02:23 | 1:59:35 | 4:00:14 | 5:59:54 | 8:00:48 | 12:00:54 | 16:01:10 | 24:01:57 | 30:00:05 | 36:00:29 | 48:00:07 |
| 5 | Group 2 | 1:01:17 | 2:00:17 | 3:59:54 | 5:59:37 | 8:01:13 | 12:00:21 | 16:00:19 | 24:01:11 | 29:59:58 | 36:03:08 | 48:00:05 |
| 6 | Lot no. | 1:00:15 | 1:59:56 | 3:59:38 | 6:00:26 | 8:00:27 | 12:00:28 | 15:59:48 | 24:00:16 | 29:59:45 | 36:02:34 | 47:59:58 |
| 7 | 00953-051 | 1:00:04 | 1:59:37 | 3:59:40 | 6:00:03 | 8:00:07 | 12:00:13 | 15:59:29 | 23:59:48 | 29:59:53 | 36:01:11 | 46:00:17 |
| 8 | 12-h | 0:59:32 | 2:01:37 | 4:00:22 | 5:59:30 | 8:00:07 | 11:59:44 | 16:00:30 | 23:59:33 | 30:00:48 | 36:01:30 | 48:00:20 |
| 9 | Group 3 | 0:59:21 | 2:01:02 | 3:59:29 | 5:59:38 | 7:59:38 | 12:00:00 | 15:59:39 | 23:59:36 | 30:00:00 | 36:00:35 | 47:59:39 |
| 10 | Lot no. | 0:59:32 | 2:00:00 | 3:59:17 | 5:59:33 | 7:59:32 | 12:00:38 | 15:59:29 | 23:59:29 | 30:00:06 | 36:00:00 | 47:59:59 |
| 11 | 00953-070 | 0:59:21 | 2:01:58 | 3:59:41 | 5:59:36 | 7:59:51 | 11:59:44 | 15:59:52 | 23:59:33 | 29:59:41 | 36:00:37 | 47:59:33 |
| 12 | 20-h | 0:59:37 | 2:01:56 | 4:01:13 | 5:59:25 | 8:00:16 | 12:02:36 | 15:59:39 | 24:00:26 | 30:00:21 | 38:02:54 | 47:59:38 |

Levels of both liothyronine (T3) and tetraiodothyronine (T4) were measured. Results from the group of dogs given the 8-hour controlled release tablets, Group 1, are shown in Tables 14 and 15. Results from the group of dogs given the 12-hour controlled release tablets, Group 2, are shown in Tables 16 and 17. Results from the group of dogs given the 20-hour controlled release tablets, Group 3, are shown in Tables 18 and 19.

TABLE 14
Concentrations of T3 (ng/ml) in Serum of Male Dogs Given a Single 50 μg Oral Tablet
Dose 8-hour Controlled Release Tablet (Group 1)
T3 Concentrations (ng/mL)

| | Blood Sample Collection Time (h) | | | | | | | | | | | |
|---|---|---|---|---|---|---|---|---|---|---|---|---|
| Subject | Baseline | 1 | 2 | 4 | 6 | 8 | 12 | 16 | 24 | 30 | 36 | 48 |
| 1 | 1.02* | 1.62 | 2.94* | 2.72* | 2.05* | 1.62* | 1.32* | 1.01* | 0.997 | 0.901 | 0.884 | 1.11* |
| 2 | 0.842* | 0.791 | 1.17* | 1.12* | 1.13 | 0.966 | 1.05 | 0.999 | 1.11 | 1.00 | 0.811 | 0.933 |
| 3 | 0.859* | 0.906* | 1.50* | 2.15* | 1.57 | 1.17* | 1.06* | 0.777* | 0.814 | 1.05 | 0.998 | 1.05 |
| 4 | 0.917 | 1.39* | 3.65* | 2.35 | 2.09 | 1.24 | 0.947* | 1.01* | 1.30 | 1.21 | 1.07 | 0.934 |
| Mean | 0.91 | 1.18 | 2.32 | 2.08 | 1.71 | 1.25 | 1.09 | 0.95 | 1.05 | 1.04 | 0.94 | 1.01 |
| SD | 0.07 | 0.34 | 1.02 | 0.59 | 0.39 | 0.24 | 0.14 | 1.10 | 0.17 | 0.11 | 0.10 | 0.07 |
| % CV | 7.7 | 28.9 | 43.9 | 28.4 | 22.9 | 19.0 | 12.5 | 10.4 | 16.6 | 10.6 | 10.5 | 7.4 |
| n | 4 | 4 | 4 | 4 | 4 | 4 | 4 | 4 | 4 | 4 | 4 | 4 |

*indicates serum appeared hemolyzed

TABLE 15
Concentrations of T4 (ng/ml) in Serum of Male Dogs Given a Single 50 μg Oral Tablet
Dose 8-hour Controlled Release Tablet (Group 1)
T4 Concentrations (ng/mL)

| | Blood Sample Collection Time (h) | | | | | | | | | | | |
|---|---|---|---|---|---|---|---|---|---|---|---|---|
| Subject | Baseline | 1 | 2 | 4 | 6 | 8 | 12 | 16 | 24 | 30 | 36 | 48 |
| 1 | 28* | 29 | 35* | 35* | 30* | 24* | 15* | 10* | 13 | 19 | 13 | 22* |
| 2 | 30* | 21 | 23* | 23* | 25 | 17 | 15 | 17 | 13 | 22 | 16 | 22 |
| 3 | 27* | 22* | 26* | 28* | 28 | 23* | 16* | 15* | 17 | 28 | 23 | 28 |
| 4 | 18 | 13* | 10* | 14 | 12 | 11 | BQL* | BQL* | BQL | 18 | 14 | 12 |
| Mean | 26 | 21 | 24 | 25 | 24 | 19 | 15 | 14 | 14 | 22 | 17 | 21 |
| SD | 5 | 6 | 9 | 8 | 7 | 5 | 0 | 3 | 2 | 4 | 4 | 6 |
| % CV | 17.9 | 26.7 | 38.1 | 30.6 | 29.5 | 27.8 | 3.1 | 21.0 | 13.2 | 17.9 | 23.7 | 27.4 |
| n | 4 | 4 | 4 | 4 | 4 | 4 | 4 | 4 | 4 | 4 | 4 | 4 |

*indicates serum appeared hemolyzed
BQL = below quantitation limit

TABLE 16
Concentrations of T3 (ng/ml) in Serum of Male Dogs Given a Single 50 μg Oral Tablet
Dose 12-hour Controlled Release Tablet (Group 2)
T3 Concentrations (ng/mL)

| | Blood Sample Collection Time (h) | | | | | | | | | | | |
|---|---|---|---|---|---|---|---|---|---|---|---|---|
| Subject | Baseline | 1 | 2 | 4 | 6 | 8 | 12 | 16 | 24 | 30 | 36 | 48 |
| 5 | 1.11 | 1.07 | 0.943 | 1.02 | 1.12 | 1.10 | 1.08* | 1.05 | 1.04 | 0.859 | 0.826 | 0.958 |
| 6 | 0.851 | 0.761* | 1.19* | 0.998* | 0.911 | 0.857 | 0.735* | 0.712* | 0.727 | 0.989 | 0.710 | 0.849 |
| 7 | 1.08* | 0.800 | 1.27 | 0.991 | 0.969 | 0.860 | 0.895 | 0.984 | 0.811 | 0.810 | 0.833 | 0.944 |
| 8 | 1.19 | 1.13* | 1.27 | 1.39* | 1.18 | 1.08 | 1.08* | 1.18* | 1.43 | 0.894 | 1.17* | 0.936* |
| Mean | 1.058 | 0.941 | 1.168 | 1.100 | 1.044 | 0.975 | 0.949 | 0.982 | 1.002 | 0.888 | 0.885 | 0.922 |
| SD | 0.126 | 0.162 | 0.134 | 0.168 | 0.109 | 0.116 | 0.145 | 0.172 | 0.272 | 0.066 | 0.173 | 0.043 |
| % CV | 11.9 | 17.3 | 11.4 | 15.3 | 10.4 | 11.9 | 15.3 | 17.5 | 27.2 | 7.4 | 19.5 | 4.6 |
| n | 4 | 4 | 4 | 4 | 4 | 4 | 4 | 4 | 4 | 4 | 4 | 4 |

*indicates serum appeared hemolyzed

TABLE 17
Concentrations of T4 (ng/ml) in Serum of Male Dogs Given a Single 50 μg Oral Tablet
Dose 12-hour Controlled Release Tablet (Group 2)
T4 Concentrations (ng/mL)

| | Blood Sample Collection Time (h) | | | | | | | | | | | |
|---|---|---|---|---|---|---|---|---|---|---|---|---|
| Subject | Baseline | 1 | 2 | 4 | 6 | 8 | 12 | 16 | 24 | 30 | 36 | 48 |
| 5 | 22 | 27 | 12 | 16 | 22 | 21 | 14* | 15 | 18 | 18 | 16 | 16 |
| 6 | 21 | 11* | 29* | 25* | 20 | 16 | 13* | 10* | 14 | 24 | 14 | 15 |
| 7 | 23* | 17 | 17 | 13 | 12 | 11 | 17 | 20 | 19 | 20 | 20 | 25 |
| 8 | 21 | 17* | 20 | 22* | 23 | 21 | 14* | 11* | ND | BQL | 24* | BQL* |
| Mean | 22 | 18 | 20 | 19 | 19 | 17 | 15 | 14 | 17 | 21 | 19 | 19 |
| SD | 1 | 6 | 6 | 5 | 4 | 4 | 2 | 4 | 2 | 2 | 4 | 4 |
| % CV | 3.8 | 31.9 | 31.7 | 25.0 | 22.5 | 24.0 | 10.3 | 28.1 | 12.7 | 12.1 | 20.8 | 24.1 |
| n | 4 | 4 | 4 | 4 | 4 | 4 | 4 | 4 | 4 | 4 | 4 | 4 |

*indicates serum appeared hemolyzed
ND = no data
BQL = below quantination limit

TABLE 18
Concentrations of T3 (ng/ml) in Serum of Male Dogs Given a Single 50 μg Oral Tablet
Dose 20-hour Controlled Release Tablet (Group 3)
T3 Concentrations (ng/ml)

| | Blood Sample Collection Time (h) | | | | | | | | | | | |
|---|---|---|---|---|---|---|---|---|---|---|---|---|
| Subject | Baseline | 1 | 2 | 4 | 6 | 8 | 12 | 16 | 24 | 30 | 36 | 48 |
| 9 | 0.757 | 0.813* | 0.877 | 0.757 | 0.702 | 0.701 | 0.861* | 0.710 | 0.676 | 0.714 | 0.746 | 0.690 |
| 10 | 1.05 | 0.84* | 1.17 | 1.44 | 1.28 | 0.987 | 0.899* | 0.815 | 0.850 | 0.917 | 0.873 | 0.811 |
| 11 | 0.890* | 0.744* | 1.23 | 0.871 | 0.727 | 0.780 | 0.731* | 0.661* | 0.595 | 0.589 | 0.659 | 0.817 |
| 12 | 1.03 | 1.06 | 1.05 | 0.964* | 0.897 | 0.807 | 1.07* | 0.973 | 0.954 | 0.794 | 0.969* | 0.983 |
| Mean | 0.932 | 0.863 | 1.081 | 1.008 | 0.902 | 0.819 | 0.890 | 0.790 | 0.769 | 0.754 | 0.812 | 0.825 |
| SD | 0.118 | 0.117 | 0.134 | 0.260 | 0.232 | 0.105 | 0.121 | 0.120 | 0.141 | 0.119 | 0.118 | 0.104 |
| % CV | 12.7 | 13.5 | 12.4 | 25.8 | 25.7 | 12.8 | 13.6 | 15.1 | 18.4 | 15.8 | 14.6 | 12.6 |
| n | 4 | 4 | 4 | 4 | 4 | 4 | 4 | 4 | 4 | 4 | 4 | 4 |

*indicates serum appeared hemolyzed

TABLE 19
Concentrations of T4 (ng/ml) in Serum of Male Dogs Given a Single 50 μg Oral Tablet
Dose 20-hour Controlled Release Tablet (Group 3)
T4 Concentrations (ng/mL)

| | Blood Sample Collection Time (h) | | | | | | | | | | | |
|---|---|---|---|---|---|---|---|---|---|---|---|---|
| Subject | Baseline | 1 | 2 | 4 | 6 | 8 | 12 | 16 | 24 | 30 | 36 | 48 |
| 9 | 19 | 28* | 27 | 24 | 23 | 20 | 25* | 16 | 20 | 23 | 23 | 22 |
| 10 | 23 | 20* | 24 | 31 | 30 | 26 | 19* | 17 | 17 | 25 | 20 | 17 |
| 11 | 25* | 27* | 29 | 25 | 22 | 23 | 18* | 23* | 23 | 22 | 22 | 29 |
| 12 | 17 | 31 | 31 | 26* | 22 | 21 | 27* | 22 | 24 | 23 | 27* | 28 |
| Mean | 21 | 27 | 28 | 27 | 24 | 23 | 22 | 20 | 21 | 23 | 23 | 24 |
| SD | 3 | 4 | 3 | 3 | 3 | 2 | 4 | 3 | 3 | 1 | 3 | 5 |
| % CV | 15.1 | 15.2 | 9.3 | 10.2 | 13.8 | 10.2 | 17.2 | 15.6 | 13.0 | 4.7 | 11.1 | 20.2 |
| n | 4 | 4 | 4 | 4 | 4 | 4 | 4 | 4 | 4 | 4 | 4 | 4 |

*indicates serum appeared hemolyzed

In-vivo Tests of Tablets of Formulation L and M

Additional studies were done to determine the blood concentration of liothyronine (T3) in dogs using tablets of Formulation L and M. In this study, four groups of three male beagle dogs were each given a Cytomel® Tablet or tablet of Formulation L or M made in accordance with the present invention, as shown in Table 20. Group 1 was given an 25 μg immediate release (“IR”) tablet. Group 2 was given an 50 μg immediate release tablet. Group 3 was given an 8-hour controlled release tablet (Formulation L) and Group 4 was given a 12-hour controlled release tablet (Formulation M).

TABLE 20

| Group No. | No. of Animals | Formulation | Route | Dose (μg) |
|---|---|---|---|---|
| 1 | 3 | IR Tablet | Oral | 25 |
| 2 | 3 | IR Tablet | Oral | 50 |
| 3 | 3 | 8 hr MR Tablet | Oral | 50 |
| | | (Formulation G) | | |
| 4 | 3 | 12 hr MR Tablet | Oral | 50 |
| | | (Formulation H) | | |

Blood samples were taken prior to the administration of any tablets in order to establish a baseline. Then blood samples were then taken at 1, 2, 4, 6, 8, 12, 16, 24, 30, 36, and 48 hours. The concentrations of T3 in serum of male beagle dogs are shown in Tables 21-24.

TABLE 21
Concentrations of T3 in Serum of Male Beagle Dogs Given a 25 μg
IR Tablet Dose of T3

| Time | T3 Concentration (ng/mL) | | | | | |
|---|---|---|---|---|---|---|
| (h) | Animal 1 | Animal 2 | Animal 3 | Mean | SD | % CV |
| 0 | 0.709 | 1.040 | 1.037 | 0.929 | 0.190 | 20 |
| 1 | 1.119 | 1.213 | 0.998 | 1.110 | 0.108 | 10 |
| 2 | 0.769 | 1.037 | 0.858 | 0.888 | 0.136 | 15 |
| 4 | 1.744 | 0.783 | 0.918 | 1.148 | 0.520 | 45 |
| 6 | 0.831 | 1.260 | 0.777 | 0.956 | 0.265 | 28 |
| 8 | 1.038 | 1.294 | 0.872 | 1.068 | 0.213 | 20 |
| 12 | 1.134 | 0.752 | 1.012 | 0.966 | 0.195 | 20 |
| 16 | 1.466 | 0.975 | 1.035 | 1.159 | 0.268 | 23 |
| 24 | 1.739 | 1.969 | 1.095 | 1.601 | 0.453 | 28 |
| 30 | 2.469 | 1.102 | 2.981 | 2.184 | 0.971 | 44 |
| 36 | 2.377 | 1.213 | 1.033 | 1.541 | 0.730 | 47 |
| 48 | 1.034 | 0.736 | 0.955 | 0.908 | 0.154 | 17 |

SD = standard deviation
% CV = percent coefficient of variation

TABLE 22
Concentrations of T3 in Serum of Male Beagle Dogs Given a 50 μg
IR Tablet Dose of T3

| Time | T3 Concentration (ug/mL) | | | | | |
|---|---|---|---|---|---|---|
| (h) | Animal 4 | Animal 5 | Animal 6 | Mean | SD | % CV |
| 0 | 0.919 | 0.698 | 0.789 | 0.802 | 0.111 | 14 |
| 1 | 5.137 | 1.077 | 4.409 | 3.541 | 2.165 | 61 |
| 2 | 3.993 | 1.017 | 2.966 | 2.659 | 1.512 | 57 |
| 4 | 2.439 | 1.001 | 2.085 | 1.842 | 0.749 | 41 |
| 6 | 1.656 | 0.754 | 1.733 | 1.381 | 0.544 | 39 |
| 8 | 1.255 | 0.742 | 1.550 | 1.182 | 0.409 | 35 |
| 12 | 0.837 | 0.787 | 1.018 | 0.881 | 0.122 | 14 |
| 16 | 0.699 | 0.660 | 0.887 | 0.749 | 0.121 | 16 |
| 24 | 0.809 | 0.652 | 0.718 | 0.726 | 0.079 | 11 |
| 30 | 0.887 | 0.670 | 0.824 | 0.794 | 0.112 | 14 |
| 36 | 0.855 | 0.826 | 0.975 | 0.885 | 0.079 | 9 |
| 48 | 0.890 | 0.732 | 0.711 | 0.778 | 0.098 | 13 |

SD = standard deviation
% CV = percent coefficient of variation

TABLE 23
Concentrations of T3 in Serum of Male Beagle Dogs Given a 50 μg
8 h Modified Release Tablet Dose of T3

| Time | T3 Concentration (ng/mL) | | | | | |
|---|---|---|---|---|---|---|
| (h) | Animal 7 | Animal 8 | Animal 9 | Mean | SD | % CV |
| 0 | 0.885 | 0.828 | 0.922 | 0.878 | 0.047 | 5 |
| 1 | 0.946 | 0.989 | 0.897 | 0.944 | 0.046 | 5 |
| 2 | 1.049 | 1.047 | 0.773 | 0.956 | 0.159 | 17 |
| 4 | 1.115 | 0.834 | 0.958 | 0.969 | 0.141 | 15 |
| 6 | 0.983 | 0.946 | 1.096 | 1.008 | 0.078 | 8 |
| 8 | 1.262 | 0.896 | 1.396 | 1.185 | 0.259 | 22 |
| 12 | 1.102 | 0.819 | 1.759 | 1.227 | 0.482 | 39 |
| 16 | 1.092 | 1.046 | 2.287 | 1.475 | 0.704 | 48 |
| 24 | 0.950 | 0.722 | 0.898 | 0.857 | 0.119 | 14 |
| 30 | 1.074 | 1.156 | 1.353 | 1.194 | 0.143 | 12 |
| 36 | 0.897 | 0.893 | 1.485 | 1.092 | 0.341 | 31 |
| 48 | 0.818 | 0.598 | 1.018 | 0.811 | 0.210 | 26 |

SD = standard deviation
% CV = percent coefficient of variation

TABLE 24
Concentrations of T3 in Serum of Male Beagle Dogs Given a 50 μg
12 h Modified Release Tablet Dose of T3

| Time | T3 Concentration (ng/mL) | | | | | |
|---|---|---|---|---|---|---|
| (h) | Animal 10 | Animal 11 | Animal 12 | Mean | SD | % CV |
| 0 | 0.880 | 0.973 | 0.902 | 0.918 | 0.049 | 5 |
| 1 | 0.913 | 1.249 | 0.927 | 1.030 | 0.190 | 18 |
| 2 | 1.365 | 1.247 | 1.103 | 1.238 | 0.131 | 11 |
| 4 | 2.868 | 1.181 | 1.011 | 1.687 | 1.027 | 61 |
| 6 | 2.759 | 1.241 | 0.846 | 1.615 | 1.010 | 63 |
| 8 | 1.727 | 1.061 | 0.847 | 1.212 | 0.459 | 38 |
| 12 | 1.335 | 1.316 | 0.906 | 1.186 | 0.242 | 20 |
| 16 | 1.025 | 1.165 | 0.828 | 1.006 | 0.169 | 17 |
| 24 | 0.921 | 1.438 | 0.923 | 1.094 | 0.298 | 27 |
| 30 | 0.813 | 1.111 | 0.882 | 0.935 | 0.156 | 17 |
| 36 | 0.946 | 1.125 | 0.852 | 0.974 | 0.139 | 14 |
| 48 | 0.792 | 0.911 | 0.807 | 0.837 | 0.065 | 8 |

SD = standard deviation
% CV = percent coefficient of variation

Pharmacokinetic Study of T3 Administered Orally to Male Beagle Dogs

Solid-phase ¹²⁵I radioimmunoassay methods were used for the quantitative determination of human total T3 concentration in dog serum samples from the in-vivo studies of Formulation L and M.

Standards, quality control samples, non-specific binding controls and unknown samples were assayed in duplicate tubes for each run. The determined non-compartmental pharamacokinetic parameters are shown in Table 25.

TABLE 25
Cumulative T3 Formulation Results after Oral Gavage to Beagle Dogs

| | Baseline | | | AUC | AUC_{BC} |
|---|---|---|---|---|---|
| 50 μg Dose | concentration | Cₘₐₓ | Fold | (ng/ | (ng/ |
| Formulation | (ng/mL) | (ng/mL) | increase | mL · hr) | mL · hr) |
| IR-1 | 0.92 | 5.14 | 5.6 | 55.1 | 21.5 |
| | 0.70 | 1.08 | 1.5 | 36.2 | 5.01 |
| | 0.79 | 4.41 | 5.6 | 53.9 | 19.9 |
| Mean (SD) | | | 4.2 | 48.4 | 15.5 |
| | | | (2.3) | (10.5) | (9.1) |
| IR-2 | 1.03 | 4.54 | 4.4 | 63.9 | 19.7 |
| | 1.01 | 3.21 | 3.2 | 57.7 | 9.25 |
| | 0.71 | 4.27 | 6.0 | 50.1 | 16.1 |
| Mean (SD) | | | 4.5 | 57.2 | 15.0 |
| | | | (1.4) | (6.9) | (5.3) |
| 8-HR-1 | 0.89 | 1.26 | 1.4 | 47.9 | 8.62 |
| | 0.83 | 1.16 | 1.4 | 42.3 | 13.6 |
| | 0.92 | 2.29 | 2.5 | 65.5 | 28.5 |
| Mean (SD) | | | 1.8 | 51.9 | 16.9 |
| | | | (0.6) | (12.1) | (10.3) |
| 8-HR-2 | 1.19 | 1.48 | 1.2 | 56.8 | 10.3 |
| | 1.13 | 1.79 | 1.6 | 53.6 | 15.2 |
| | 0.64 | 1.16 | 1.8 | 40.5 | 15.6 |
| Mean (SD) | | | 1.5 | 50.3 | 13.7 |
| | | | (0.3) | (8.6) | (3.0) |
| 12-HR-1 | 0.88 | 2.87 | 3.3 | 55.9 | 18.0 |
| | 0.97 | 1.44 | 1.5 | 56.2 | 12.5 |
| | 0.90 | 1.10 | 1.2 | 42.1 | 3.26 |
| Mean (SD) | | | 2.0 | 51.4 | 11.3 |
| | | | (1.1) | (8.1) | (7.4) |
| 12-HR-2 | 0.70 | 1.15 | 1.6 | 47.6 | 17.9 |
| | 0.98 | 3.05 | 3.1 | 50.0 | 16.1 |
| | 1.23 | 1.48 | 1.2 | 61.2 | 12.3 |

| | | | |
|---|---|---|---|
| Mean (SD) | 2.0 | 52.9 | 15.4 |
| | (1.0) | (7.3) | (2.9) |

AUC values were estimated using the trapezoid rule and truncating at 48 hrs (Cₗₐₛₜ)
AUC_{BC} values (background corrected) were determined by subtracting off the lowest concentration observed from all other plasma values during the time course and using the trapezoid rule truncating at 48 hrs to estimate the actual exposure above endogenous T3

Both modified release formulations mitigate the concentration peak (Cₘₐₓ) observed with the 50 μg immediate release (IR) formulation. The overall mean exposures from the 50 μg 8-HR doses are essentially equivalent to the overall mean IR formulation.

The overall mean exposures from the 50 μg 12-HR doses are approximately 90% of the exposures determined for the overall mean IR formulation.

While the present invention may be embodied in many different forms, several embodiments are discussed herein with the understanding that the present disclosure is to be considered only as an exemplification of the principles of the invention, and it is not intended to limit the invention to the embodiments described or illustrated.

What is claimed is:

1. A sustained release, oral pharmaceutical composition comprising: liothyronine or a pharmaceutically acceptable salt thereof wherein prior to 2 hours after administration of the pharmaceutical composition, the plasma concentration of liothyronine does not exceed the baseline concentration of liothyronine by more than 3.5 times that of the baseline concentration and wherein the administration of the composition reduces the frequency or eliminates the occurrence of an adverse cardiac effect.

2. The pharmaceutical composition of claim 1, wherein the adverse cardiac effect is one or more of the following symptoms selected from the group consisting of fluctuations in heart rate, fast or irregular heartbeat, heart palpitations increased blood pressure, increased risk of heart attack, chest pain, and congestive heart failure.

3. The pharmaceutical composition of claim 1, wherein prior to 1 hour after administration of the pharmaceutical composition, the plasma concentration of liothyronine does not exceed the baseline concentration of liothyronine by more than 3.5 times that of the baseline concentration.

4. The pharmaceutical composition of claim 1, wherein upon administration, the plasma concentration of liothyronine does not exceed the baseline concentration by more than 3 times that of the baseline concentration.

5. The pharmaceutical composition of claim 1, wherein, upon administration, the plasma concentration of liothyronine does not exceed the baseline concentration by more than 2.5 times that of the baseline concentration.

6. The pharmaceutical composition of claim 1, wherein, upon administration, the plasma concentration of liothyronine does not exceed the baseline concentration by more than 2 times that of the baseline concentration.

7. The pharmaceutical composition of claim 1, wherein, upon administration, the plasma concentration of liothyronine does not exceed the baseline concentration by more than 1.5 times that of the baseline concentration.

8. The pharmaceutical composition of claim 1, wherein the pharmaceutical composition exhibits a zero-order release rate of liothyronine or a pharmaceutically acceptable salt thereof.

9. The pharmaceutical composition of claim 1, wherein the pharmaceutical composition exhibits a first-order release rate of liothyronine or a pharmaceutically acceptable salt thereof.

10. The pharmaceutical composition of claim 1, wherein the pharmaceutically acceptable salt of liothyronine is liothyronine sodium.

11. The pharmaceutical composition of claim 1, wherein the amount of liothyronine or a pharmaceutically acceptable salt thereof is about 0.01% to 0.1% by weight.

12. The pharmaceutical composition of claim 1, wherein the amount of liothyronine or a pharmaceutically acceptable salt thereof is about 0.05% to 0.6% by weight.

13. The pharmaceutical composition of claim 1, wherein the liothyronine or a pharmaceutically acceptable salt thereof is released over a period of 8 hours or more.

14. The pharmaceutical composition of claim 1, wherein the liothyronine or a pharmaceutically acceptable salt thereof is released over a period of 12 hours or more.

15. The pharmaceutical composition of claim 1, wherein the liothyronine or a pharmaceutically acceptable salt thereof is released over a period of 24 hours or more.

16. The pharmaceutical composition of claim 1, wherein 80 percent of the liothyronine or a pharmaceutically acceptable salt thereof is released in about 8, 12, 20, or 24 hours.

17. The pharmaceutical composition of claim 1, wherein about 75 to about 90 percent of the liothyronine or a pharmaceutically acceptable salt thereof is released in about 24 hours or more.

18. The pharmaceutical composition of claim 1, wherein about 85 percent of the liothyronine or a pharmaceutically acceptable salt thereof is released in about 24 hours.

19. The pharmaceutical composition of claim 1 further comprising a rate-limiting matrix.

20. The pharmaceutical composition of claim 19, wherein the rate-limiting matrix is a cellulose based polymer.

21. The pharmaceutical composition of claim 20, wherein the cellulose based polymer is hydroxypropyl methyl cellulose, hydroxymethyl cellulose, ethyl cellulose or a combination thereof.

22. The pharmaceutical composition of claim 19, wherein the amount of the rate-limiting matrix is about 20% to 60% by weight of the pharmaceutical composition.

23. The pharmaceutical composition of claim 19, wherein the amount of the rate-limiting matrix is about 30% to 50% by weight of the pharmaceutical composition.

24. The pharmaceutical composition of claim 19, wherein the amount of the rate-limiting matrix is about 40% by weight of the pharmaceutical composition

25. The pharmaceutical composition of claim 19 further comprising a filler, a glidant, a lubricant, a binder, a disintegrate or a combination thereof.

26. The pharmaceutical composition of claim 25, wherein the filler is microcrystalline cellulose.

27. The pharmaceutical composition of claim 25, wherein the glidant is silicone dioxide.

28. The pharmaceutical composition of claim 25, wherein the lubricant is stearic acid.

29. The pharmaceutical composition of claim 1, wherein the pharmaceutical composition is a tablet or capsule.

30. A sustained release, oral pharmaceutical composition comprising: liothyronine or a pharmaceutically acceptable salt thereof wherein after 1 hour post administration of the pharmaceutical composition, the plasma concentration of liothyronine does not fluctuate more than 65% per hour.

31. The pharmaceutical composition of claim 30, wherein the concentration of liothyronine does not fluctuate more than 50% per hour.

32. The pharmaceutical composition of claim 30, wherein the concentration of liothyronine does not fluctuate more than 40% per hour.

33. The pharmaceutical composition of claim 30, wherein the concentration of liothyronine does not fluctuate more than 30% per hour.

34. The pharmaceutical composition of claim 30, wherein the concentration of liothyronine does not fluctuate more than 10% per hour.

35. The pharmaceutical composition of claim 30, wherein the liothyronine or a pharmaceutically acceptable salt thereof is released over a period of 8 hours or more.

36. The pharmaceutical composition of claim 30, wherein the liothyronine or a pharmaceutically acceptable salt thereof is released over a period of 12 hours or more.

37. The pharmaceutical composition of claim 30, wherein the liothyronine or a pharmaceutically acceptable salt thereof is released over a period of 24 hours or more.

38. The pharmaceutical composition of claim 30, wherein the pharmaceutically acceptable salt of liothyronine is liothyronine sodium.

39. The pharmaceutical composition of claim 30 further comprising a rate-limiting matrix.

40. The pharmaceutical composition of claim 39, wherein the rate-limiting matrix is a cellulose based polymer.

41. The pharmaceutical composition of claim 40, wherein the cellulose based polymer is hydroxypropyl methyl cellulose, hydroxymethyl cellulose, ethyl cellulose or a combination thereof.

42. The pharmaceutical composition of claim 39, wherein the amount of the rate-limiting matrix is about 20% to 60% by weight of the pharmaceutical composition.

43. The pharmaceutical composition of claim 39, wherein the amount of the rate-limiting matrix is about 30% to 50% by weight of the pharmaceutical composition.

44. The pharmaceutical composition of claim 39, wherein the amount of the rate-limiting matrix is about 40% by weight of the pharmaceutical composition.

45. The pharmaceutical composition of claim 39 further comprising a filler, a glidant, a lubricant, a binder, a disintegrate or a combination thereof.

46. The pharmaceutical composition of claim 45, wherein the filler is microcrystalline cellulose.

47. The pharmaceutical composition of claim 45, wherein the glidant is silicone dioxide.

48. The pharmaceutical composition of claim 45, wherein the lubricant is stearic acid.

49. The pharmaceutical composition of claim 39, wherein the pharmaceutical composition is a tablet or capsule.

50. A sustained release, oral pharmaceutical composition comprising:
(a) liothyronine or a pharmaceutically acceptable salt thereof; and
(b) hydroxypropyl methyl cellulose, wherein the amount of hydroxypropyl methyl cellulose is about 30% to 70% by weight, and wherein the Cₘₐₓ of liothyronine occurs at least 1 hour after administration.

51. The pharmaceutical composition of claim 50, wherein Cmax occurs at least 2 hours after administration.

52. The pharmaceutical composition of claim 50, wherein Cmax occurs at least 3 hours after administration.

53. The pharmaceutical composition of claim 50, wherein the pharmaceutical composition exhibits a zero-order release rate of liothyronine or a pharmaceutically acceptable salt thereof.

54. The pharmaceutical composition of claim 50, wherein the pharmaceutical composition exhibits a first-order release rate of liothyronine or a pharmaceutically acceptable salt thereof.

55. The pharmaceutical composition of claim 50, wherein the pharmaceutically acceptable salt of liothyronine is liothyronine sodium.

56. The pharmaceutical composition of claim 50, wherein the amount of liothyronine or a pharmaceutically acceptable salt thereof is about 0.01 % to 0.1 % by weight.

57. The pharmaceutical composition of claim 50, wherein the amount of liothyronine or a pharmaceutically acceptable salt thereof is about 0.05% to 0.6% by weight.

58. The pharmaceutical composition of claim 50 further comprising a filler, a glidant, a lubricant, a binder, a disintegrate or a combination thereof.

59. The pharmaceutical composition of claim 58, wherein the filler is microcrystalline cellulose.

60. The pharmaceutical composition of claim 58, wherein the glidant is silicone dioxide.

61. The pharmaceutical composition of claim 58, wherein the lubricant is stearic acid.

62. The pharmaceutical composition of claim 61, wherein the pharmaceutical composition is a tablet or capsule.

63. A sustained release, oral pharmaceutical composition comprising:
(a) liothyronine or a pharmaceutically acceptable salt thereof in the amount of about 1.25 μg to 100 μg;
(b) hydroxypropylmethylcellulose, wherein the amount of hydroxypropylmethylcellulose is about 40-60% by weight;
(c) silicone dioxide;
(d) microcrystalline cellulose; and
(e) stearic acid.

64. The pharmaceutical composition of claim 63 further comprising calcium sulfate.

65. The pharmaceutical composition of claim 63, wherein the liothyronine or a pharmaceutically acceptable salt thereof is present in the amount of about 25 μg to 75 μg.

66. The pharmaceutical composition of claim 63, wherein the liothyronine or a pharmaceutically acceptable salt thereof is present in the amount of about 50 μg.

67. A method of treating thyroid hormone deficiency comprising administering to an individual the pharmaceutical pharmaceutical composition of claim 1.

68. A method of treating thyroid hormone deficiency comprising administering to an individual the pharmaceutical pharmaceutical composition of claim 63.
